(19) United States
(12) Patent Application Publication
(10) Pub. No.: US 2011/0136935 A1
(43) Pub. Date: Jun. 9, 2011

(54) BONE AND/OR DENTAL CEMENT COMPOSITION AND USES THEREOF

(75) Inventors: Eugene Khor, Singapore (SG); Chang Ming Guo, Singapore (SG); Hong Wu, Singapore (SG); Lee Yong Lim, Western Australia (AU)

(73) Assignees: NATIONAL UNIVERSITY OF SINGAPORE, (SG); SINGAPORE HEALTH SERVICES PTE LTD, (SG)

(21) Appl. No.: 12/675,424

(22) PCT Filed: Aug. 30, 2007
(86) PCT No.: PCT/SG07/00287

§ 371 Date: Feb. 26, 2010

(51) Int. Cl. A61K 6/083; A61Q 11/00

(52) U.S. Cl. 523/116; 523/115

(57) ABSTRACT

The present invention provides a composition comprising: at least one viscosity-enhancing agent; and at least one macromonomer. In particular, the composition may be a bone cement composition and/or a dental cement composition. The composition may be used in medicine. The composition may be used in orthopedic and/or periodontal applications. The present invention also provides uses of the composition.

### FIELD OF THE INVENTION

The present invention relates to a composition comprising at least one viscosity-enhancing agent and at least one macromonomer. In particular, the composition is a bone cement composition and/or a dental cement composition. The present invention also provides uses of the composition.

### BACKGROUND OF THE INVENTION

Injectable bone cement, mainly poly(methyl methacrylate) (PMMA) and calcium phosphate cement (CPC), have been clinically utilized for more than 20 years as bone repairing materials. Injectable bone cement is a paste formed before it is used in the body of a subject but solidifies pretty rapidly once it is injected into the human body. From the perspective of both clinical observation and laboratory experimental results, these two injectable bone cements exhibit their own significant advantages. However, there also exist several problems during surgical procedures as well as post-operative complications.

PMMA is an injectable bone cement used extensively in applications such as percutaneous injectable vertebroplasty & kyphoplasty for the reconstruction of spinal fracture, fixation of total hip replacement, bony void fillers, etc. The quick setting time and strong final mechanical strength make this material popular in the operation theatre. However, several complications have manifested with deeper comprehension of this material. The problems associated with PMMA bone cement include high exothermic temperature during hardening that can lead to osteonecrosis, residual toxic methyl methacrylate monomer, non-osteoconductivity, the solidified cement is very stiff and there is poor interaction between the implant with host bone, just to name a few. Some efforts have been made to eliminate these drawbacks. Unfortunately, its intrinsic properties such as the increase in temperature during polymerization and toxic monomer cannot be avoided.

In the case of CPC, two obvious advantages of this cement are its osteoconducting property and the fact that no heat is generated during the setting of CPC. Eventually the calcium phosphate compound encourages growth of new bone tissue around the implanted cement. The serenity of the hardening process eliminates the potential risk of osteonecrosis of PMMA. However, there are several disadvantages associated with CPC such as ready disintegration of the cement after it is injected at the bone site and poor injectability due to its poor paste quality leading to liquid-solid phase separation.

Many efforts have been made to ameliorate the problems of formulations containing PMMA or CPC as described above. Most approaches comprise the incorporation of a variety of other substances targeting specific performance function limitations of the base PMMA or CPC cement formulations. For example, polysaccharide derivatives such as dextran sulfate aqueous solution (30 to 60 w/w %) was introduced into calcium phosphate cement to achieve a moderate viscosity material (U.S. Pat. No. 5,993,535); phosphorylated chitin and chitosan was incorporated into CPC to reduce disintegration of the cement (CN1470247A). Polyacrylic acid was also used as lubricant liquids to make cement paste with reactive tricalcium phosphate nanoparticles (US 2006/0118007 A1). The combination of PMMA and hydroxyapatite to form binary compositions to enhance the biological activity of PMMA cement has been proposed as a polymer cement for percutaneous vertebroplasty (US 20050256220A1). However, these formulations are only somewhat effective and not all the problems described above are solved.

New formulations that mimic the PMMA and CPC have also been proposed. For example, chitosan, malic acid and hydroxyapatite mixture have been mixed to produce an osteoconductive bone filling material (U.S. Pat. No. 5,618,339). U.S. Pat. No. 6,124,373 describes bone replacement material comprising biodegradable and biocompatible poly(propylene fumurate) and inorganic fillers which present lower hardening temperature with necessary compressive strength.

Therefore, there is a need in the art for a suitable composition with fewer drawbacks and better performance characteristics.

### SUMMARY OF THE INVENTION

The present invention seeks to address the problems above, and provide a composition suitable for use in several applications such as in orthopedic and/or periodontal applications. In particular, the composition may be used as a bone cement composition and/or a dental cement composition.

According to a first aspect, the present invention provides a composition comprising:
(a) at least one viscosity-enhancing agent; and
(b) at least one macromonomer.

Any suitable viscosity-enhancing agent may be used for the purposes of the present invention. The viscosity-enhancing agent may have an average molecular weight equal to or greater than 5000 Da. For example, the viscosity-enhancing agent may be derivatives of chitin, chitosan, cellulose, dextran, collagen, hyaluronic acid and/or starch, or a combination thereof. In particular, the viscosity-enhancing agent is chitin methacrylate. The chitin methacrylate may have a molecular weight of about 15000 Da. Chitin methacrylate has a general structure as follows:

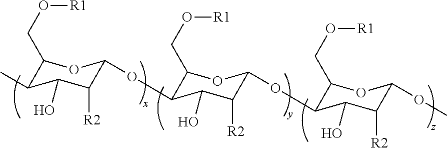

wherein:
x, y, and z are integers and each x, y or z is ≧1;
each R₁ is the same or different, selected from the group consisting of:

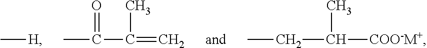

wherein M is a metal; and
each R₂ is the same or different, selected from the group consisting of:

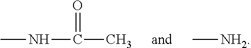

M may be any suitable metal. In particular, M is any monoelectropositive metal. Even more in particular, M is sodium (Na) or potassium (K). In particular, at least one R₁ in the structure is:

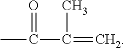

In an alternative embodiment, at least one R₁ in the structure above comprises an unsaturated functional group. For example, the unsaturated functional group may be a functional group with a carbon-carbon double bond or a carbon-carbon triple bond. In particular, at least one R₁ in the structure comprises a carbon-carbon double bond. Even more in particular, at least one R₁ in the structure is:

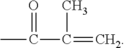

Any suitable macromonomer may be used for the purposes of the present invention. For example, the macromonomer may be a cross-linking macromonomer. The macromonomer may be a polymer with at least one double bond. In particular, the macromonomer is polyethylene glycol) diacrylate (PEGDA), copolymers thereof, or a combination thereof.

According to a particular aspect, the present invention may further comprise at least one solvent. Any suitable solvent may be used. For example, the solvent may be any one of, or a combination of, water, ethanol and saline. In particular, the solvent is water.

According to another particular aspect, the present invention may further comprise at least one osteoconductive material. Any suitable osteoconductive material may be used for the purposes of the present invention. The osteoconductive material may be a calcium-containing inorganic compound. In particular, the osteoconductive material is calcium hydroxyapatite.

Another particular aspect of the present invention provides the composition according to any aspect further comprising a radiopaque material. Any suitable radiopaque material may be used. For example, the radiopaque material may be an oxide or halogen salt of a heavy metal. The heavy metal may be any one of, or a combination of, gold, barium, silver and bismuth. In particular, the radiopaque material is barium sulfate.

According to another particular aspect, the composition may further comprise at least one polymerization initiator and/or at least one polymerization accelerator. Any suitable polymerization initiator and/or polymerization accelerator may be used for the purposes of the present invention. For example, the polymerization initiator may be ammonia persulfate (APS), potassium persulfate (KPS) or a mixture thereof. In particular, the polymerization initiator is ammonia persulfate. The polymerization accelerator may be N,N,N′,N′-Tetramethylethylenediamine (TEMED).

The composition may further comprise at least one polymerization inhibitor. Any suitable polymerization inhibitor may be used. For example, the polymerization inhibitor may be hydroquinone, p-benzoquinone, trinitrobenzene, nitrobenzene or diphenylpicrylhydrazyl (DPPH). In particular, the polymerization inhibitor is hydroquinone.

The composition may further comprise at least one bioactive agent. For example, the bioactive agent may include, but is not limited to proteins, antibiotics, therapeutic agents such as anti-tumour agents and chemotherapeutic agents, vitamins, growth factors, cells, or a combination thereof. The proteins may be bone morphogenetic proteins. The cells may be adult stem cells that have been induced into osteoblasts.

The composition according to any aspect of the present invention may be a bone cement composition and/or a dental cement composition. The composition may be used for any suitable application. The composition may be used for therapeutic and/or non-therapeutic applications. The composition according to any aspect of the present invention may be for use in medicine. The composition according to any aspect of the present invention may be for use in periodontal and/or orthopedic applications. The composition according to any aspect of the present invention may also be for use in dentistry.

For example, the composition according to any aspect may be for use in bone filling, bone repairing, bone implanting, for joining bone implants, pulp capping, root perforation repair, root-end filling, apical barrier, root fracture, bleaching and/or temporary filling. The composition according to any aspect of the present invention may be for use as bone void filler, bone cement, dental filler material, fixation cement for bone prosthesis implantation and/or filler of soft tissue spaces.

According to another aspect, the present invention provides a use of at least one viscosity-enhancing agent and at least one macromonomer, as defined above, for the preparation of a composition for medical treatment. The composition may be a bone cement composition and/or a dental cement composition. Medical treatment may comprise orthopedic and/or periodontal applications.

The present invention also provides a method of bone filling, bone repairing, bone implanting and/or joining bone implants, comprising applying a composition according to any aspect of the present invention to a bone site of a subject. The method may be a therapeutic or non-therapeutic method.

The present invention also provides a method of pulp capping, root perforation repair, root-end filling, management of root fracture, root canal filling and/or temporary filing, wherein the method comprises applying a composition according to any aspect of the present invention to a tooth and/or gum of a subject. The method may be a therapeutic or non-therapeutic method.

According to another aspect, the present invention provides a bone implant comprising the composition according to any aspect of the present invention. The bone implant may be used for any suitable application. For example, the bone implant may be used for therapeutic or non-therapeutic applications.

Another aspect of the present invention is a kit comprising at least one viscosity-enhancing agent and at least one macromonomer, as defined above. The kit may optionally further comprise: at least one solvent; at least one osteoconductive material; at least one radiopaque material; at least one polymerization initiator; at least one polymerization accelerator; at least one polymerization inhibitor; and/or at least one bioactive agent. The solvent, osteoconductive material, radiopaque material, polymerization initiator, polymerization accelerator, polymerization inhibitor and/or bioactive agent may be as described above.

The present invention also provides a kit comprising a composition according to any aspect of the present invention.

The kits according to any aspect of the present invention may further comprise a set of instructions on the use of the kit.

The present invention also provides a compound having the formula:

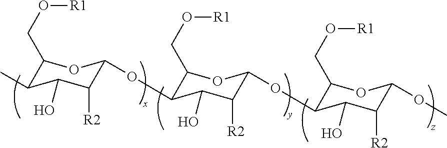

wherein:
x, y, and z are integers and each x, y or z is ≧1;
each R₁ is the same or different, selected from the group consisting of:

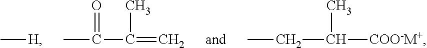

wherein M is a metal; and
each R₂ is the same or different, selected from the group consisting of:

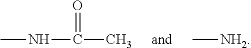

M may be any suitable metal. In particular, M is any monoelectropositive metal. Even more in particular, M is sodium (Na) or potassium (K). In particular, at least one R₁ in the structure is:

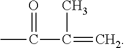

According to an alternative embodiment, at least one R₁ in the compound comprises an unsaturated functional group. For example, the unsaturated functional group may be a functional group with a carbon-carbon double bond or a carbon-carbon triple bond. In particular, at least one R₁ in the compound, comprises a carbon-carbon double bond. Even more in particular, at least one R₁ in the compound is:

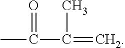

The present invention also provides stereoisomers of the compound according to any aspect of the invention.

The compound may be chitin methacrylate. In particular, the compound may have a structure as follows:

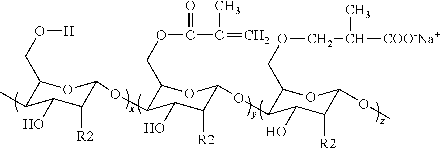

wherein:
x, y, and z are integers and each x, y or z is ≧1; and
each R₂ is the same or different, selected from the group consisting of:

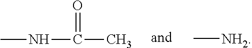

The compound may have an average molecular weight from about 10000 Da to about 15000 Da. The compound may have a degree of N-acetylation equal to or greater than 50%. For example, the degree of N-acetylation may be about 60%, 70%, 75%, 80%, 85%, 90%, 95%, 98%, 100%. In particular, the degree of N-acetylation may be greater than or equal to 80%. Even more in particular, the degree, of N-acetylation is 90%.

Even more in particular, the compound may have a structure as follows:

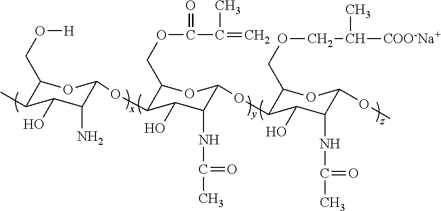

wherein x, y, and z are integers and each x, y or z is ≧1. The above structure is a non-limiting example. Accordingly, other structures may also be possible, depending on the degree of N-acetylation.

The compound according to any aspect of the present invention may be soluble in some solvents. Examples of solvents in which the compound may be soluble include, but are not limited to, polar solvents, such as methanol, ethanol and propanol, and, water-based solvents.

The compound may also be in the form of a hydrogel when the compound is cross-linked. Accordingly, when the hydrogel is contacted with suitable solvents, such as, but not limited to, polar solvents, such as methanol, ethanol and propanol, and water-based solvents, the hydrogel may swell.

The compound may be photocrosslinkable. The compound may undergo polymerization. For example, the compound may be polymerized by any suitable method. In particular, the compound may be polymerized under UV-irradiation. Even more in particular, the compound may be polymerized in the presence of the at least one polymerization initiator and/or at least one polymerization accelerator as described above.

According to a particular aspect, the compound may be for use in medicine. The compound may be for use in at least one of the following: orthopedic applications, periodontal applications, biomedical applications and dentistry. For example, the compound may be for use in drug delivery, as wound dressing, as bone substitute, in bone cement composition, in dental cement composition and/or as skin substitute, soft tissue repairing and/or replacement materials. In particular, the compound may be for use in drug delivery. The compound may be a drug carrier and/or a drug delivery agent.

According to another aspect, the present invention provides a use of a compound according to any aspect of the invention in the manufacture of a composition for drug delivery. The composition may further comprise at least one macromonomer. Any suitable macromonomer may be used, as described above. In particular, the macromonomer is poly(ethylene glycol) diacrylate (PEGDA), copolymers thereof, and/or a combination thereof.

The present invention also provides a method of delivering at least one bioactive agent to a subject comprising the steps of:
(a) providing at least one bioactive agent and a compound according to any aspect of the present invention;
(b) loading the bioactive agent to the compound; and
(c) administering the bioactive agent loaded compound to the subject.

The bioactive agent may be any suitable bioactive agent. For example, the bioactive agent may be as described above.

The present invention also provides a drug delivery agent and/or a drug carrier comprising a compound according to any aspect of the invention. The drug delivery agent and/or drug carrier may further comprise at least one macromonomer. Any suitable macromonomer may be used. For example, the macromonomer may be as described above.

Another aspect of the present invention is a hydrogel comprising the compound according to any aspect as described above. The hydrogel may further comprise at least one macromonomer. Any suitable macromonomer may be used. For example, the macromonomer may be as described above. The hydrogel may be used in various applications. According to a particular aspect, the hydrogel may be for use in biomedical applications. The hydrogel may be for use in drug delivery, in wound dressing, as bone substitute, in bone cement composition, as temporal joint spacers, in dental cement composition, as skin substitute, in soft tissue distension, in soft tissue repairing and/or as replacement materials, as a drug carrier and/or as a drug delivery agent.

The present invention also provides a composition comprising the compound according to any aspect of the invention. The composition may further comprise at least one macromonomer. Any suitable macromonomer may be used. For example, the macromonomer may be as described above. The composition may further comprise at least one solvent; at least one osteoconductive material; at least one radiopaque material; at least one polymerization initiator; at least one polymerization accelerator; at least one polymerization inhibitor; and/or at least one bioactive agent. Any suitable solvent, osteoconductive material, radiopaque material, polymerization initiator, polymerization accelerator, polymerization inhibitor and/or bioactive agent may be used, for example as described above.

According to a, particular aspect, the composition may be a bone cement composition and/or a dental cement composition.

According to another particular aspect, the composition may be for use in various applications. For example, the composition may be for use in medicine. The composition may be for use in dentistry. The composition may be for use in orthopedic and/or periodontal applications.

The present invention also provides a method of preparing the compound according to any aspect of the present invention. The method comprises the steps of:
(a) depolymerization of chitin; and
(b) esterification of the product from step (a) with methacrylic acid.

### BRIEF DESCRIPTION OF THE FIGURES

FIG. 1: The dynamic exothermal curve during solidification (setting) of composition according to one aspect of the invention with three different amounts of polymerization initiator (“a”) and polymerization accelerator (“t”) in 37° C. water bath.

FIG. 2: The cells viability in direct contact with samples. The columns are plotted as the optical density to the incubation time of each sample (n=6). The composition according to the present invention is indicated as “cement”. Polystyrene film was used as the negative control while rubber latex glove was used as the positive control.

FIG. 3: The cells viability when in indirect contact (extract) with samples. The column is plotted as the optical density to the incubation time of each sample (n=6). The composition according to the invention is indicated as “cement extract”.

FIG. 4: X-ray images of five specimens of the composition according to the present invention comprising different ratios of hydroxyapatite and barium sulfate in solid powder part. The values 0%, 10%, 20%, 30% and 40% represent the % weight of barium sulfate in the total mass of hydroxyapatite and barium sulfate.

FIG. 5: (A), (B) and (C) represent the visualizing ability of the composition according to the present invention under fluoroscope when the composition is injected into cadaveric spine.

FIG. 6: Segments of the gel permeation chromatography (GPC) of six low molecular weight chitin samples.

FIG. 7: Fourier transform infrared (FTIR) spectra of (a) hydrolyzed chitin and (b) methacrylated chitin.

FIG. 8: ¹H-NMR of chitin methacrylate in D₂O.

FIG. 9: ¹³C-NMR of chitin methacrylate in D₂O.

FIG. 10: Sample of low molecular weight chitin methacrylate (LCMA) hydrogel.

FIG. 11: Thermograms of (a) chitin, (b) depolymerized chitin and (c) LCMA—curves a1, b1, c1 indicate the weight loss curves and curves a2, b2 and c2 indicate the derivative weight loss curves.

FIG. 12: X-ray diffraction patterns of (a) chitin, (b) low molecular weight (LMW) chitin and (c) LCMA.

FIG. 13: Degradation rate of hydrogel in lysosome solution.

FIG. 14: Percentage of cell viability of direct assay contact.

FIG. 15: SEM photomicrographs of cell morphology on methacrylated chitin hydrogel—(a) CCL-1 on day 2; (b) CCL-186 on day 2; (c) CRL-1427 on day 2; (d) CCL-1 on day 5; (e) CCL-186 on day 5; (f) CRL-1427 on day 5.

FIG. 16: Swelling kinetic curves of P0L5 in pH 3 and pH 7 media.

FIG. 17: DSC thermograms of pure water and hydrogels P0L5, P5L5 and P10L5.

FIG. 18: SEM photographs of LCMA and LCMA/PEGDA hydrogels—(a1) P0L5 hydrogel (x200); (a2) P0L5 hydrogel (x500); (b1) P5L5 hydrogel (x200); (b2) P5L5 hydrogel (x500); (c1) P10L5 hydrogel (x200); (c2) P10L5 hydrogel (x500).

FIG. 19: Vitamin B₁₂ release profile of xerogels (in Milli-Q water).

FIG. 20: Vitamin B₁₂ release profile of xerogels (in 0.1M HCl).

FIG. 21: Vitamin B₁₂ release profile of hydrogels (in Milli-Q water).

FIG. 22: Vitamin B₁₂ release profile of hydrogels (in 0.1M HCl).

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

The present invention provides a composition for use in several applications. For example, the composition may, be used as bone cement composition and/or dental cement composition. The composition may be used for therapeutic or non-therapeutic applications. The applications of the composition may include, but is not limited to, orthopedic and/or periodontal applications. The composition may be an injectable composition. The setting time of the composition may make the composition particularly suitable for use in several applications.

According to a first aspect, the present invention provides a composition comprising:
(a) at least one viscosity-enhancing agent; and
(b) at least one macromonomer.

Any suitable macromonomer may be used for the present invention. A macromonomer may be referred to as a macromer, and therefore, these terms may be used interchangeably. For the purposes of the present invention, a macromonomer will be defined as a polymer or oligomer whose molecules each have a polymerizable functional group, often at the end that enables it to act as a monomer. After polymerization, the groups are part of the main chain of the final polymer.

The at least one macromonomer may be about 20-50 weight % based on the total weight of the composition. The at least one macromonomer may be a cross-linking macromonomer. The macromonomer may be a polymer with at least one double bond. The at least one macromonomer may be a functional macromonomer with unsaturated groups. The macromonomer may be miscible in water. The macromonomer may rapidly polymerize in the presence of at least one polymerization initiator and/or at least one polymerization accelerator. The at least one macromonomer may reduce the solidification temperature of the composition as there are less unsaturated groups in terms of the same mass of monomer. In particular, the at least one macromonomer may be poly(ethylene glycol) diacrylate (PEGDA), copolymers thereof and/or a combination thereof. PEGDA may be a suitable macromonomer as PEGDA has good biocompatibility (D Wang et al, 2004).

Any suitable viscosity-enhancing agent may be used. The at least one viscosity-enhancing agent may be about 0.1-10 weight % based on the total weight of the composition. The viscosity-enhancing agent may be water-soluble. The at least one viscosity-enhancing agent may enhance the cohesion of the composition. In particular, the viscosity-enhancing agent may enhance the cohesion of the bone cement composition and/or dental cement composition of the present invention. The at least one viscosity-enhancing agent may have an average molecular weight equal to or greater than 5000 Da. For example, the average molecular weight may be 6500 Da or more, 8000 Da pr more, 10000 Da or more. In particular, the average molecular weight of the viscosity-enhancing agent is about 15000 Da. For example, the molecular weight may be estimated as M_{w} by gel permeation chromatography (GPC) using pullulan as a standard. The higher the molecular weight of the viscosity-enhancing agent, the less soluble in water it will be. The at least one viscosity-enhancing agent may be a polysaccharide or a derivative thereof.

The at least one viscosity-enhancing agent may be selected from the group consisting of: derivatives of chitin, chitosan, cellulose, dextran, collagen, hyaluronic acid, starch, and a combination thereof. In particular, the viscosity-enhancing agent may be selected from the group consisting of: chitin methacrylate, dextran methacrylate, chitosan itaconylate, carboxymethyl chitin, methacrylate hyaluronan, poly(vinyl alcohol), polyethylene oxide, carboxymethylcellulose, hydroxypropylmethylcellulose, derivatives thereof, and a mixture thereof. Even more in particular, the at least one viscosity-enhancing agent is chitin methacrylate. Chitin methacrylate will be described in more detail below.

According to a particular aspect, the composition may be a bone cement composition and/or a dental cement composition.

The composition according to any aspect of the present invention may further comprise at least one solvent. The at least one solvent may be about 15-30 weight % based on the total weight of the composition. Any suitable solvent may be used for the purposes of the present invention. For example, the solvent may include, but is not limited to, water, ethanol, saline, or a combination thereof. In particular, the solvent is water. The viscosity-enhancing agent may be mixed with the solvent. Therefore, the compatibility of the viscosity-enhancing agent with the solvent may be important.

The composition according to any aspect of the present invention may further comprise at least one osteoconductive material. The at least one osteoconductive material may be about 20-50 weight % based on the total weight of the composition. For the purposes of the present invention, an osteoconductive material is one which leads to bone deposition. An osteoconductive material is one which provides a platform to allow and/or promote new bone growth, and enhance the mechanical strength at mean time. The osteoconductive material may be similar to natural bone mineral, which comprises calcium phosphate salt with different crystal phases. Any suitable osteoconductive material may be used for the present invention. The osteoconductive material of the present invention may be a calcium-containing inorganic compound. In particular, the osteoconductive material may be selected from the group consisting of: calcium hydroxyapatite, calcium phosphate salt, calcium sulfate, calcium carbonate, strontium and a mixture thereof. The calcium phosphate salt may be β-tricalcium phosphate. Even more in particular, the at least one osteoconductive material is calcium hydroxyapatite.

Hydroxyapatite is osteoconductive because it is able to adsorb osteogenic growth factors from the local milieu and from the circulation, thus creating suitable conditions for bone formation when implanted in an osseous environment. Hydroxyapatite is a calcium phosphate compound and it is the most common composition in natural bone mineral. Synthetic hydroxyapatite with less than 20 μm granule size is preferred for use in the present invention as it shows almost the same biological and physicochemical properties to natural bone mineral. The easy fabrication and synthesis of synthetic hydroxyapatite also makes it more obtainable than natural bone hydroxyapatite.

According to a particular aspect of the present invention, the composition may further comprise at least one radiopaque material. Any suitable radiopaque material may be used. The radiopaque material may be about 4-15 weight % based on the total weight of the composition. The radiopaque material may be an oxide or halogen salt of a heavy metal. Examples of heavy metals include gold, barium, silver, bismuth or a combination thereof. However, any other suitable heavy metal may be used. Examples of radiopaque materials include, but are not limited to, barium sulfate, tungsten, bismuth compounds, tantalum, zirconium, platinum, gold, silver, stainless steel, titanium, alloys thereof, combinations thereof, or other equivalent materials for use as radiographic agents. In particular, the radiopaque material may be bismuth oxide, bismuth trioxide, barium sulfate or a mixture thereof. The bismuth oxide may also provide the colour to the composition. Other examples of radiopaque material include ZrO₂ and BaO. Even more in particular, the radiopaque material is barium sulfate.

The advantage of adding a radiopaque material to the composition of the present invention is that the composition will be able to display radiopacity to enable visualisation and assessment in a radiograph. This may be useful in diagnostics. Depending on the degree of radiopaqueness desired, various ratios of a radiopaque material may be added.

The composition according to any aspect of the present invention may further comprise at least one polymerization initiator and/or at least one polymerization accelerator. The at least one polymerization initiator and/or polymerization accelerator may initiate a polymerization reaction and may result in the formation of a composition which may be ready for use, such as a bone cement composition and/or dental cement composition. The at least one polymerization accelerator may be used with at least one polymerization initiator to catalyse the polymerization of the at least one macromonomer comprised in the composition. Any suitable polymerization initiator and/or polymerization accelerator may be used for the purposes of the present invention. The polymerization initiator may be about 0.1-0.2 weight % based on the total weight of the composition. The polymerization accelerator may be about 0.08-0.16 weight % based on the total weight of the composition.

The at least one polymerization initiator may be a water soluble redox initiator. In particular, the polymerization initiator may be: ammonia persulfate (APS), potassium persulfate (KPS) and/or a mixture thereof. Even more in particular, the at least one polymerization initiator is ammonium persulfate (APS).

The at least one polymerization accelerator may be N,N,N′,N′-tetramethylethylenediamine (TEMED), N,N-dihydroxyethyl-p-toluidine (DHEPT), N-phenylglycine, β-dimethylaminoprionitrile, sodium meta bisulfate, potassium meta bisulfate, and/or a combination thereof. In particular, the at least one polymerization accelerator is be N,N,N′,N′-tetramethylethylenediamine (TEMED).

The composition may further comprise at least one polymerization inhibitor. The at least one polymerization inhibitor may prevent premature crosslinking of the composition. In particular, the polymerization inhibitor may prevent or reduce the premature generation of free radicals which lead to premature crosslinking. Such premature crosslinking may render the composition useless. Any suitable polymerization inhibitor may be used. For example, any suitable chemical or material which prevents free-radical polymerization may be used for the purposes of the present invention. The at least one polymerization inhibitor may delay or prevent crosslinking during storage, manufacture and/or transport of the composition. For example, the polymerization inhibitor may be hydroquinone, p-benzoquinone, trinitrobenzene, nitrobenzene or diphenylpicrylhydrazyl (DPPH). In particular, the polymerization inhibitor is hydroquinone. Only a small amount of polymerization inhibitor is required. For example, the polymerization inhibitor may be about 0.01-0.1 weight % based on the total weight of the composition.

The setting time of the composition may vary depending on the amounts of polymerization initiator and/or polymerization accelerator used. For the purposes of the present invention, setting time will be defined as the time required for the composition to reach a set or solid state after being applied in a fluid or paste state. In particular, setting time refers to the time required for the composition to attain a specified degree of rigidity. The setting times may be varied by the amount of solvent or the ratio of materials used. Accordingly, the setting time of the composition may be controlled by adjusting the amounts of polymerization initiator and/or polymerization accelerator used in the composition. The setting time may also be dependent on the surrounding or environmental temperature at which the composition is being polymerized. This will be explained in more detail below. For example, for the same amounts of polymerization initiator and polymerization accelerator used, the setting time of the composition at 25° C. is more than double than at 37° C. It should also be noted that heat may be released during the polymerization process. The amount of heat released during the polymerization process may depend on the amount of polymerization initiator and/or polymerization accelerator added to the composition. The mixing temperature may also be varied to change the polymerization time.

A skilled person would realize that for some applications, a shorter setting time is desired, whereas for other applications, a longer setting time is desired. For example, in orthopedic surgical applications, a long setting time may be required to allow the surgeon to have sufficient time to load and/or inject the composition to the appropriate bone site of the patient. The setting time of the composition according to any aspect of the present invention may be from 1 minute to 1 hour. In particular, the setting time may be from 4 minutes to 45 minutes, from 5 minutes to 40 minutes, from 6 minutes to 35 minutes, from 7 minutes to 30 minutes, from 8 minutes to 25 minutes, from 9 minutes to 20 minutes, from 10 minutes to 15 Minutes, from 11 minutes to 13 minutes. Even more in particular, the setting time is from 4 minutes to 7 minutes.

The composition according to any aspect of the present invention may further comprise at least one bioactive agent. For the purposes of the present invention, a bioactive agent is defined as being any substance intended for use in the diagnosis, cure, mitigation, treatment or prevention of disease or in the enhancement of desirable physical or mental development and conditions in a subject. Any suitable bioactive agent may be used. For example, the at least one bioactive agent may include a pharmacologically active substance that produces a local and/or systemic effect in animals, preferably mammals, or humans. The at least one bioactive agent may have osteoinductive features to ameliorate bone growth to which the composition is applied and surrounding tissue growth.

For example, the at least one bioactive agent may be bone morphogenetic proteins (BMP), antibiotics, therapeutic agents such as anti-tumour agents and a chemotherapeutic agents, vitamins, cells, and/or growth factors. In particular, the BMP may be selected from the group consisting of: BMP2, BMP4, and a combination thereof. The growth factor may be selected from the group consisting of: platelet-derived growth factor (PDGF), vascular endothelial growth factor (VEGF), transforming growth factor-β (TGF-β), insulin derived growth factor (IGF), fibroblast growth factor (FGF) and a combination thereof. Any suitable antibiotic may be used. The antibiotic may be bactericidal or bacteriostatic. For example, the antibiotic may be selected from the group consisting of: gentamicin, tobramycin, penicillin antibiotics such as ampicillin, amoxicillin, penicillin G, carbenicillin, tacarcillin and methicillin, cephalosporin antibiotics such as calaclor, cefarodxil, cefamandole, cefazolin and cefaperazone, aztreonam, imipenem, macrolide antibiotics such as erythromycin, aminoglycoside antibiotics such as streptomycin, neomycin, lincomycin, kanamycin, vancomycin, sisomycin, polymixin antibiotics such as colistin, and polypeptide antibiotics such as bacitracin and novobiocin, and a combination thereof. The vitamins may be selected from the group consisting of: vitamins A, C, D, E, K, B₁, B₂, B₅, B₆, B₁₂ and a combination thereof. The cells may be adult or embryonic stem cells that have been induced into osteoblasts. In particular, the at least one bioactive agent is at least one BMP. BMPs are naturally osteoconductive and can effectively facilitate biological bone fracture healing process.

The composition according to any aspect of the present invention may be a bone cement composition and/or a dental cement composition.

The present invention also provides a method of preparing a composition of the invention, comprising the steps of:
a) providing at least one viscosity-enhancing agent and at least one macromonomer; and
b) mixing the viscosity-enhancing agent and macromonomer with at least one solvent to form a liquid component.

The method may further comprise the step of:
c) providing at least one osteoconductive material and/or at least one radiopaque material to form a powder component.

According to a particular aspect, the at least one viscosity-enhancing agent and the at least one macromonomer may be mixed in the at least one solvent to form a liquid component. The liquid component may be viscous. The liquid component may be in the form of a gel. The ratio of the at least one viscosity-agent, at least one solvent and the at least one macromonomer in the liquid component, by weight, may be in the range from 0.5:5:10 to 0.5:15:20. In particular, the range is from 0.5:8:10 to 1:12:17. More in particular, the ratio is 1:10:15. Even more in particular, the ratio of chitin methacrylate:water:poly(ethylene glycol)diacrylate is 1:10:15.

According to a particular aspect, the at least one osteoconductive material and/or the at least one radiopaque material may be mixed to form a powder component of the composition. In particular, the ratio of the at least one osteoconductive material to the at least one radiopaque material may be from 95:5 to 60:40. In particular, the ratio is from 90:10: to 80:20. Even more in particular, the ratio of hydroxyapatite to barium sulfate is from 90:10: to 80:20. At such a range, the powder component may be clear enough to be viewed under X-ray and still able to maintain its mechanical strength when used as part of the composition for various applications.

The liquid component from step b) may be the composition. The liquid component from step b) may be used for various applications as described below. However, if additional properties, including, but not limited to radiopacity and/or osteoconductivity is required from the composition, the powder component from step c) may be mixed with the liquid component from step b).

The liquid component and powder component may be mixed together to form a paste. For example, the ratio of the liquid component to the powder component, by weight, may be in the range of 5:1 to 1:1. In particular, the range is 2:1 to 1:1. Even more in particular, the ratio is 1.3:1.

According to a particular aspect, the powder component is mixed with the liquid component. The composition comprising the powder component and the liquid component may be in the form of a paste. A polymerization initiator and/or a polymerization accelerator may be added to the composition. Upon addition to the composition, polymerization begins and the composition hardens. The hardened composition may be useful in many applications. For a short period of time, during the polymerization, the entire composition may be doughy or workable so that the composition may form into the shape and size desired for injection, implantation or use.

The composition according to any aspect of the present invention may have different setting times, as described above. The setting time of the composition may be varied depending on the amounts of polymerization initiator and/or polymerization accelerator used. The setting time may be varied by the amount of solvent or the ratio of materials used. The mixing temperature may also be varied to change the polymerization time. According to a particular aspect, the composition hardens rapidly at body temperature. For example, body temperature may be defined as being at about 37° C. According to another particular aspect, the composition according to any aspect of the present invention may be at body temperature before use or pre-heated to about body temperature before use. Alternatively, if it is not desired for the composition to harden before use, the composition may be maintained at a temperature lower than body temperature during use so that the composition does not harden and the setting time is prolonged. Once it is desired for the composition to harden, the composition may be heated to about body temperature. For example, when the composition is applied to a bone site of a subject, the temperature at which the composition is applied may be at a temperature lower than body temperature. Once the composition has been applied and it is desired for the composition to harden, heat may be applied to raise the temperature of the site to about body temperature so that the composition may harden. Alternatively, the composition may be prepared at a temperature lower than body temperature so that the composition does not harden prior to being used and/or before the composition is applied to a site of a subject. The composition prepared may be cooled to a temperature lower than body temperature so that the composition does not harden prior to being used and/or before the composition is applied to a site of a subject.

The composition according to any aspect of the present invention may be for use in medicine. In particular, the composition is a bone cement composition and/or a dental cement composition. For example, the composition may be used as bone void filler, bone cement, dental filler material, fixation cement for bone prosthesis implantation and/or filler of soft tissue spaces.

The composition may be for use in orthopedic applications. For example, the composition may be for use in arthroplasty procedures of joints, for the fixation of prostheses to living bones. The composition may be for use in bone filling, bone repairing, bone implanting and/or for joining bone implants. The composition may be for use when reconstruction is necessary because of osteoarthritis, rheumatoid arthritis, traumatic arthritis, avascular necrosis, non-union of fractures of the neck of the femur, sickle cell anemia, osteoporosis, secondary severe joint destruction following trauma or other conditions (including fixation of some unstable fractures in metastatic malignancies), collagen disease and/or revision of previous arthroplasty procedures. The composition according to any aspect of the present invention may also be used for filling bone voids and/or gaps of the skeletal system, such as extremities, craniofacial, spine and pelvis, and for bone defects. The composition may also be used for distal radius void filling, tibial plateau void filling, tibial pilon void filling and/or calcaneal void filling.

The composition according to any aspect of the present invention may be generally used for fixation of artificial joints to bone stock. The composition may be used as fixation cement for bone prosthesis implantation. The composition may act more as a filler. For example, the composition may serve as a grout or interfacial material between the reamed medullary canal of the proximal femur or tibia for total hip or knee implants, respectively, and the metallic stem of the prosthesis. The composition applied to the medullary canal, for example, is intended to form a layer (mantle) of uniform thickness between the bone and the implant stem. This cement mantle is intended to mechanically interlock with the pores of the prepared bone and structurally compensate for the inability of the surgical technique to create a cavity in bone that exactly matches the shape of the total joint stem.

For the purposes of the present invention a “gap” refers to a cavity of the bone. The term “defect” refers to a condition which may be considered a disease and needs to be treated therapeutically, whilst with the term gap, it refers to a condition which is not necessarily a disease and may be treated non-therapeutically, for example for cosmetic purposes.

The composition according to any aspect of the present invention may also be for use in vertebroplasty and/or kyphoplasty. In particular, the composition may be used as a bone cement composition for vertebroplasty and/or kyphoplasty. Vertebroplasty and kyphoplasty are recently developed techniques for treating vertebral compression fractures. Percutaneous vertebroplasty was first reported by a French group in 1987 for the treatment of painful hemangiomas. In the 1990's, percutaneous vertebroplasty was extended to indications including osteoporotic vertebral compression fractures, traumatic compression fractures, and painful vertebral metastasis. In one percutaneous vertebroplasty technique, the composition may be percutaneously injected into a fractured vertebral body via a trocar and cannula system. The targeted vertebrae are identified under fluoroscopy. A needle is introduced into the vertebral body under fluoroscopic control to allow direct visualization. A transpedicular (through the pedicle of the vertebrae) approach is typically bilateral but can be done unilaterally. The composition may also be delivered through an extrapedicular route.

Kyphoplasty is a modification of percutaneous vertebroplasty. Kyphoplasty involves a preliminary step that comprises the percutaneous placement of an inflatable balloon tamp in the vertebral body. Inflation of the balloon creates a cavity in the bone prior to composition injection. Further, the proponents of percutaneous kyphoplasty have suggested that high pressure balloon-tamp inflation can at least partially restore vertebral body height. In kyphoplasty, it has been proposed that the composition can be injected at lower pressures into the collapsed vertebra since a cavity exists to receive the composition—which is not the case in conventional vertebroplasty.

The composition according to any aspect of the present invention may also be for use in dentistry and/or periodontal applications. Dentistry and/or periodontal applications may be therapeutic dental treatments or non-therapeutic procedures. Non-therapeutic procedures may comprise cosmetic dental procedures. The composition according to any aspect of the present invention may be used for several clinical dental procedures including, but not limited to, pulp capping, management of root fracture, root perforation repair, root-end filling, barrier cement for intracoronal bleaching and/or apical barrier/plug. The composition may also be used as a temporary filling material to be placed in the tooth until a permanent restoration can be placed. The composition may also be used as a permanent filling material. The composition may further be used as a protective cement barrier in internal bleaching. The composition may be placed above the root canal filling material to minimise the leakage of bleaching agents through the root. Yet another use of the composition may be as a management of root fracture, in which the composition may be used to root fill a tooth with root fracture.

When the composition is used in pulp capping, it is used in the management of deep carious lesions, accidental mechanical pulp exposure and pulp exposure following traumatic accidents. The composition is placed over the exposed pulp and a restorative filling is then placed over it.

In the case of root perforation repair, this is carried out via an intracanal approach or using a surgical approach. The intracanal approach involves placement of the composition where the perforation is, in order to seal the perforation. The surgical approach is used to achieve the same after the intracanal approach fails or if the perforation is inaccessible through the access cavity of the tooth.

For root-end filling, it is a surgical procedure carried out to establish a seal at the root-end of the tooth. A cavity is prepared at the root end of the tooth and the composition is placed in the prepared cavity.

Apical barrier/plug is carried out in non-vital, immature permanent teeth with an open root apex. The composition is placed at the root end to create an apical plug which prevents the extrusion of root canal filling material.

Details of the procedures mentioned above may be found in any standard endodontics textbook (Pathways of the pulp. Eds Cohen S, Burns R C. 8^{th} Ed. Mosby Inc. St. Louis). Further, it would be known to a person skilled in the art how to perform the procedures.

The composition according to any aspect of the present invention may be provided to a subject in different ways. For example, the composition may be an injectable composition. Accordingly, the composition may be loaded into a syringe and injected to an appropriate site of a subject. The composition may also be applied directly to an appropriate site by using suitable apparatus, such as a spatula. Alternatively, the composition may, be loaded into a temporary or permanent bag, balloon or carrier for insertion.

According to another aspect, the present invention provides a use of at least one viscosity-enhancing agent and at least one macromonomer, as defined above, for the preparation of a composition for medical treatment. Medical treatment may include, but is not limited to periodontal applications and/or orthopedic applications. The composition may further comprise at least one solvent; at least one osteoconductive material; at least one radiopaque material; at least one polymerization initiator, at least one polymerization accelerator; at least one polymerization inhibitor; and/or at least one bioactive agent, all of which are described above. In particular, the composition may be a bone cement composition and/or a dental cement composition. The composition may be used for any suitable application. For example, the composition may be used for applications as described above.

The present invention also provides a use of at least one viscosity-enhancing agent and at least one macromonomer, as defined above, for the preparation of a composition for dentistry. The composition may further comprise at least one solvent; at least one osteoconductive material; at least one radiopaque material; at least one polymerization initiator; at least one polymerization accelerator; at least one polymerization inhibitor; and/or at least one bioactive agent, all of which are described above. In particular, the composition may be a bone cement composition and/or dental cement composition. The composition may be used for any suitable application. For example, the composition may be used for applications as described above.

According to another aspect, the present invention provides a method of bone filling, bone repairing, bone implanting and/or joining bone implants, comprising applying a composition according to any aspect of the present invention to a bone site of a subject. The present invention also provides a method of vertebroplasty and/or kyphoplasty, comprising applying a composition according to any aspect of the present invention to a bone site of a subject. The subject may be a human or non-human subject. The subject may be an animal. The subject may be a mammal. The method may also be used for other applications as described above. In particular, the composition used in the method may be a bone cement composition.

The present invention also provides a method of pulp capping, root perforation repair, root-end filling, management of root fracture, root canal filling and/or temporary filling, wherein the method comprises applying a composition according to any aspect of the present invention to at least one tooth and/or gum of a subject. The subject may be a human or nonhuman subject. The subject may be an animal. The subject may be a mammal. The method may also be used for other applications as described above. In particular, the composition used in the method may be a dental cement composition.

The methods as described above comprise the step of applying an effective amount of the composition according to any aspect of the present invention at the bone site and/or at least one tooth and/or gum area of the subject. The composition may be a bone cement composition and/or a dental cement composition. As a particular, but non-limiting example, an effective amount of the composition is applied at the site of the bone defect of the subject. The composition, when applied to the bone defect, may partially or fully restore structural integrity of the bone. The composition may function as a bone filler (or partial bone filler) or plug, to mend the bone defect. The amount of composition applied may depend on the size and nature of the bone defect, and the outcome that is sought. The composition may be applied as a paste or putty, such that the composition takes the shape of the defect.

According to another aspect, the present invention also provides a method of filling a tooth cavity using a composition according to any aspect of the present invention, comprising the steps of:
identifying the cavity of the tooth to be filled;
providing the composition; and
introducing the composition into the tooth cavity whereby the path of communication between an inner portion of the cavity and the outer surface of the tooth is sealed.

In particular, the composition may be a dental cement composition.

Another aspect of the invention is a method of treating tooth decay using the composition according to any aspect of the present invention, comprising the steps of:
identifying the decay;
removing the decay to create a cavity to be filled;
providing the composition; and
introducing the composition to the cavity whereby the path of communication between an inner portion of the cavity and the outer surface of the tooth is sealed.

In particular, the composition may be a dental cement composition.

According to another aspect, the invention provides a method of performing root canal therapy on a tooth using the composition according to any aspect of the invention, comprising the steps of:
removing a portion of the tooth to expose the root canal;
preparing the root canal to be filled; and
introducing the composition into the root canal whereby the path of communication between the root canal and the outer surface of the tooth is sealed.

In particular, the composition may be a dental cement composition. The method may be a non-therapeutic method. In particular, the method may be a cosmetic method.

Another aspect of the present invention is the use of the composition according to any aspect of the invention, for cosmetic purposes. In particular, the composition is a dental cement composition. For example, the composition may be used for sealing gaps between teeth, or external appearance of the tooth. The composition may also be used for filling soft tissue, for filling gaps in bones and in plastic surgery. The composition may also be used in elevating defects, filling voids, enhancing the prominence of certain areas such as facial features or other parts of the body.

Accordingly, an aspect of the present invention provides a method of cosmetic, non-therapeutic treatment, wherein the method comprises at least one step of using the composition according to any aspect of the invention. In particular, the composition is a bone cement composition and/or a dental cement composition.

The composition according to any aspect of the present invention may be formed into an implant or may be molded into a desired shape. The shape the composition is molded into may be in the form of the defect or gap into which the molded composition is going to be inserted. Accordingly, the present invention provides an implant comprising the composition as described above.

The implant may be a bone implant and/or a dental implant. The size, shape, thickness and/or volume of the molded implant may be controlled according to what is desired and according to the application in which it will be used.

According to another aspect, the present invention provides a method of implantation, the method comprising the steps of:
identifying a site of implantation;
providing an implant comprising a composition according to any aspect of the present invention; and introducing the implant to the site of implantation.

The composition according to any aspect of the present invention may be applied so that it directly contacts existing bone and/or tooth adjacent to, or defining, the bone and/or tooth site, or the composition may be contacting another implant, or both. The bone and/or tooth site may be a bone defect and/or tooth defect site.

The present invention also provides a kit comprising the composition according to any aspect of the present invention. The composition comprised in the kit may be used for different applications as described above. The kit may further comprise a set of instructions on the uses and applications of the composition.

According to another aspect, the present invention provides a kit comprising at least one viscosity-enhancing agent and at least one macromonomer, each of which is described above. The kit may optionally comprise any one of or a combination of: at least one solvent; at least one osteoconductive material; at least one radiopaque material; at least one polymerization initiator; at least one polymerization accelerator; at least one polymerization inhibitor; and/or at least one bioactive agent, all of which are described above. The kit may further comprise a set of instructions on the use of the kit. Each component may be packaged separately within the kit. The components of the kit may be used to prepare the composition according to any aspect of the present invention. The composition may be used in various applications as described above.

According to a particular aspect, the kits described above may be used in periodontal and/or orthopedic applications. The kits may be used in dentistry. The applications may be used for therapeutic or non-therapeutic applications.

Another aspect of the present invention is a kit comprising an implant comprising the composition according to any aspect of the present invention. The implant may be a bone implant and/or a dental implant. The composition may be a bone cement composition and/or a dental cement composition. The kit may further comprise a set of instructions on the use of the implant.

Another aspect of the present invention is a compound having the following structure:

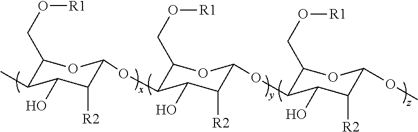

wherein:
x, y, and z are integers and each x, y or z is ≧1;
each R₁ is the same or different, selected from the group consisting of:

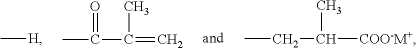

wherein M is a metal; and
each R₂ is the same or different, selected from the group consisting of:

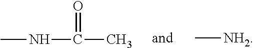

M may be any suitable metal. In particular, M may be any monoelectropositive metal. Even more in particular, M is sodium (Na) or potassium (K). In particular, at least one R₁ in the structure is:

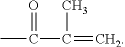

In an alternate embodiment, at least one R₁ in the structure comprises an unsaturated functional group. For example, the unsaturated functional group may be a functional group with a carbon-carbon double bond or a carbon-carbon triple bond. In particular, at least one R₁ in the structure comprises a carbon-carbon double bond. Even more in particular, at least one R₁ in the structure is:

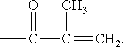

Stereoisomers of the above compound are also provided by the present invention. The compound may be referred to as chitin methacrylate. In particular, the structure of chitin methacrylate is:

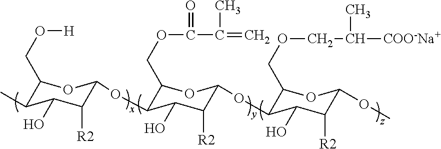

wherein:
x, y, and z are integers and each x, y or z is ≧1; and
each R₂ is the same or different, selected from the group consisting of:

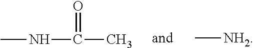

Even more in particular, the compound may have the following structure:

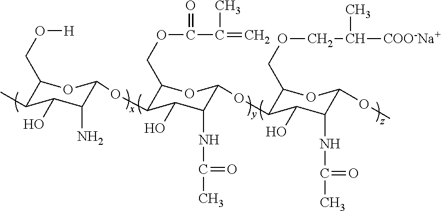

wherein x, y, and z are integers and each x, y or z is ≧1. The above structure is a non-limiting example. Accordingly, other structures may also be possible, depending on the degree of N-acetylation.

The compound of the present invention may have an average molecular weight equal to or greater than 5000 Da. In particular, the average molecular weight may be more than 8000 Da, 8500 Da, 9000 Da, 9500 Da, 10000 Da, 11000 Da, 15000 Da, 30000 Da, 50000 Da. Even more in particular, the average molecular weight is from about 10000 Da to about 15000 Da. For example, the molecular weight may be estimated as M_{w} by gel permeation chromatography (GPC) using pullulan as a standard.

The compound may have a degree of N-acetylation equal to or greater than 50%. For example, the degree of N-acetylation may be about 60%, 70%, 75%, 80%, 85%, 90%, 95%, 98%, 100%. In particular, the degree of N-acetylation may be about 90%.

The compound may have many properties. For example, the compound may be photocrosslinkable. For the purposes of the present invention, photocrosslinking will be defined as the formation of a covalent linkage between two macromolecules or between two different parts of one macromolecule. Photocrosslinking usually requires a crosslinking agent to be provided.

However, the compound of the present invention may be able to photocrosslink without the addition of a crosslinking agent.

According to a particular aspect, the compound of the present invention may undergo polymerization. The compound of the invention may undergo polymerization under specific conditions. The conditions may comprise ultraviolet (UV)-light. For example, the compound may undergo polymerization under UV light at 365 nm. The conditions may further comprise the presence of a polymerization initiator. For example, the polymerization initiator may be a photoinitiator. A photoinitiator may be a compound capable of absorbing UV light and generating an active substance which can initiate a polymerization reaction. The compound may form a hydrogel after undergoing polymerization. For the purposes of the present invention, the polymerized compound will be referred to as a hydrogel of the compound. Hydrogels are hydrophilic polymer networks that can absorb up to thousands of times their dry weight in water.

The compound of the present invention may also be dissolved in suitable solvents. For example, the solvent may be selected from the group consisting of: water, lithium chloride, dimethylacetamide (DMAc), dimethylsulfoxide (DMSO), ethanol, methanol, a aqueous polymer solution, polar solvents, and a mixture thereof. In particular, the solvent may be a mixture of lithium chloride and DMAc; ethanol and water; or methanol and water.

The compound of the present invention may be useful in different applications. The hydrogel of the compound may also be useful in different applications. According to another aspect of the present invention, the compound and/or the hydrogel may be for use in medicine and/or medical applications. The compound may be for use in orthopedic applications and/or periodontal applications. The compound may also be for use in dentistry. In particular, the compound may be for use in biomedical applications. The biomedical applications may include, but is not limited to, any one of the following: drug delivery, wound dressing, bone substitute, bone cement composition, temporal joint spacers, dental cement composition and/or skin substitute, soft tissue distension, soft tissue repairing and/or replacement materials. Even more in particular, the compound may be for use in drug delivery.

The present invention also provides a use of the compound according to any aspect of the present invention having the following formula:

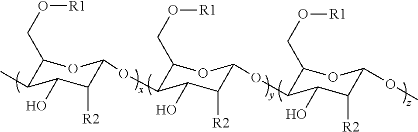

wherein:
x, y, and z are integers and each x, y or z is ≧1;
each R₁ is the same or different, selected from the group consisting of:

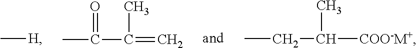

wherein M is a metal; and
each R₂ is the same or different, selected from the group consisting of:

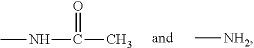

in the manufacture of a composition.

M may be any suitable metal. In particular, M is a monoelectropositive metal.

Even more in particular, M is sodium (Na) or potassium (K). In particular, at least one R₁ in the structure is:

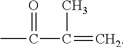

In an alternate embodiment, at least one R₁ in the compound comprises an unsaturated functional group. For example, the unsaturated functional group may be a functional group with a carbon-carbon double bond or a carbon-carbon triple bond. In particular, at least one R₁ in the compound comprises a carbon-carbon double bond. Even more in particular, at least one R₁ in the compound is:

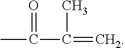

In particular, the compound may have a structure as follows:

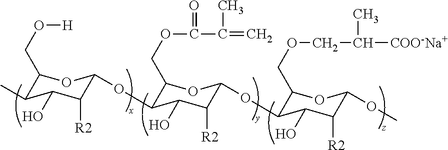

wherein:
x, y, and z are integers and each x, y or z is ≧1; and
each R₂ is the same or different, selected from the group consisting of:

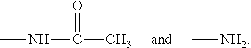

The compound may have an average molecular weight from about 10000 Da to about 15000 Da. The compound may have a degree of N-acetylation equal to or greater than 50%. For example, the degree of N-acetylation may be about 60%, 70%, 75%, 80%, 85%, 90%, 95%, 98%, 100%. In particular, the degree of N-acetylation may be greater than or equal to 80%. Even more in particular, the degree of N-acetylation is 90%.

Even more in particular, the compound may have a structure as follows:

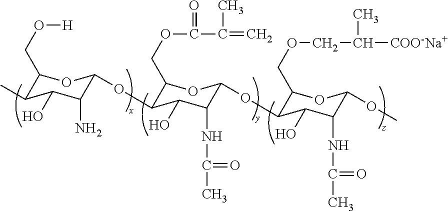

wherein x, y, and z are integers and each x, y or z is ≧1. The above structure is a non-limiting example. Accordingly, other structures may also be possible, depending on the degree of N-acetylation.

In particular, the present invention provides a use of the compound according to any aspect of the present invention in the manufacture of a composition for drug delivery. The compound may be the hydrogel of the compound. Alternatively, the composition may comprise the hydrogel of the compound. The composition may further comprise a suitable drug for delivery to a subject.

According to a particular aspect, the composition may further comprise at least one macromonomer. Any suitable macromonomer may be used. The macromonomer may be as described above. In particular, the macromonomer may be poly(ethylene glycol) diacrylate (PEGDA), copolymers thereof, and/or a combination thereof.

The present invention also provides a method of delivering at least one bioactive agent to a subject comprising the steps of:
(a) providing at least one bioactive agent and a compound according to any aspect of the present invention having the following formula:

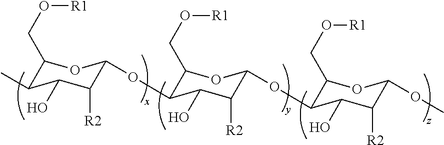

wherein:
x, y, and z are integers and each x, y or z is ≧1;
each R₁ is the same or different, selected from the group consisting of:

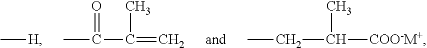

wherein M is a metal; and
each R₂ is the same or different, selected from the group consisting of:

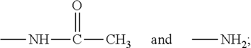

(b) loading the bioactive agent to the compound; and
(c) administering the bioactive agent loaded compound to the subject.

M may be any suitable metal. In particular, M is any monoelectropositive metal. Even more in particular, M is sodium (Na) or potassium (K). In particular, at least one R₁ in the structure is:

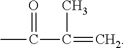

The compound may be a hydrogel of the compound.

In an alternate embodiment, at least one R₁ in the compound comprises an unsaturated functional group. For example, the unsaturated functional group may be a functional group with a carbon-carbon double bond or a carbon-carbon triple bond. In particular, at least one R₁ in the compound comprises a carbon-carbon double bond. Even more in particular, at least one R₁ in the compound is:

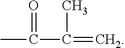

In particular, the compound may have a structure as follows:

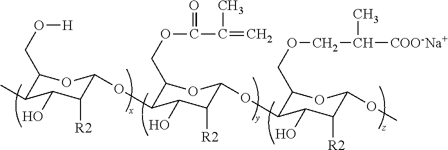

wherein:
x, y, and z are integers and each x, y or z is ≧1; and
each R₂ is the same or different, selected from the group consisting of:

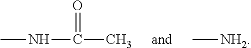

The compound may have an average molecular weight from about 10000 Da to about 15000 Da. The compound may have a degree of N-acetylation equal to or greater than 50%. For example, the degree of N-acetylation may be about 60%, 70%, 75%, 80%, 85%, 90%, 95%, 98%, 100%. In particular, the degree of N-acetylation may be greater than or equal to 80%. Even more in particular, The degree of N-acetylation is 90%.

Even more in particular, the compound may have a structure as follows:

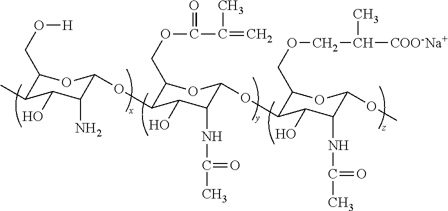

wherein x, y, and z are integers and each x, y or z is ≧1. The above structure is a non-limiting example. Accordingly, other structures may also be possible, depending on the degree of N-acetylation.

The bioactive agent may be any suitable bioactive agent. The bioactive agent may be as described above. In particular, the bioactive agent may be selected from the group consisting of: bone morphogenetic proteins (BMP), antibiotics, vitamins, growth factors, therapeutic agents, and a combination thereof. Even more in particular, the bioactive agent is vitamin B₁₂.

According to another aspect, the present invention provides a drug delivery agent and/or drug carrier comprising a compound according to any aspect of the present invention having the following formula:

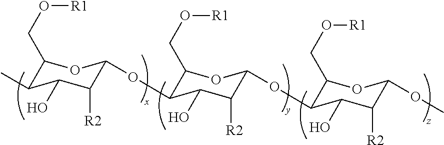

wherein:
x, y, and z are integers and each x, y or z is ≧1;
each R₁ is the same or different, selected from the group consisting of:

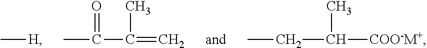

wherein M is a metal; and
each R₂ is the same or different, selected from the group consisting of:

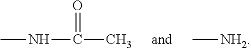

M may be any suitable metal. In particular, M is any monoelectropositive metal. Even more in particular, M is sodium (Na) or potassium (K).

In particular, at least one R₁ in the structure is:

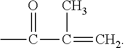

The compound may be a hydrogel of the compound.

In an alternate embodiment, at least one R₁ in the compound comprises an unsaturated functional group. For example, the unsaturated functional group may be a functional group with a carbon-carbon double bond or a carbon-carbon triple bond. In particular, at least one R₁ in the compound comprises a carbon-carbon double bond. Even more in particular, at least one R₁ in the compound is:

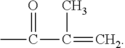

In particular, the compound may have a structure as follows:

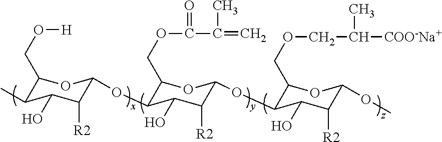

wherein:
x, y, and z are integers and each x, y or z is ≧1; and
each R₂ is the same or different, selected from the group consisting of:

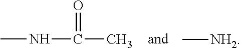

The compound may have an average molecular weight from about 10000 Da to about 15000 Da. The compound may have a degree of N-acetylation equal to or greater than 50%. For example, the degree of N-acetylation may be about 60%, 70%, 75%, 80%, 85%, 90%, 95%, 98%, 100%. In particular, the degree of N-acetylation may be greater than or equal to 80%. Even more in particular, the degree of N-acetylation is 90%.

Even more in particular, the compound may have a structure as follows:

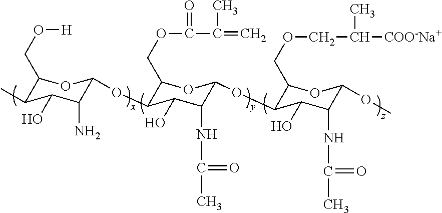

wherein x, y, and z are integers and each x, y or z is ≧1. The above structure is a non-limiting example. Accordingly, other structures may also be possible, depending on the degree of N-acetylation.

The drug delivery agent and/or drug carrier may further comprise at least one macromonomer. Any suitable macromonomer may be used. For example, the macromonomer may be as described above. In particular, the macromonomer is poly(ethylene glycol) diacrylate (PEGDA), copolymers thereof, or a combination thereof. The drug delivery agent and/or drug carrier may be loaded with at least one drug and/or at least one bioactive agent. Any suitable drug and/or bioactive agent may be used. For example, the bioactive agent may be as described above.

Another aspect of the present invention is a hydrogel comprising the compound according to any aspect of the invention having the following formula:

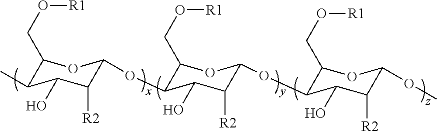

wherein:
x, y, and z are integers and each x, y or z is ≧1;
each R₁ is the same or different, selected from the group consisting of:

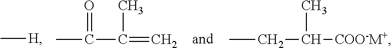

wherein M is a metal; and
each R₂ is the same or different, selected from the group consisting of:

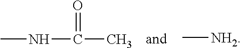

M may be any suitable metal. In particular, M is any monoelectropositive metal. Even more in particular, M is sodium (Na) or potassium (K). In particular, at least one R₁ in the structure is:

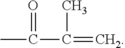

In an alternate embodiment, at least one R₁ in the compound comprises an unsaturated functional group. For example, the unsaturated functional group may be a functional group with a carbon-carbon double bond or a carbon-carbon triple bond. In particular, at least one R₁ in the compound comprises a carbon-carbon double bond. Even more in particular, at least one R₁ in the compound is:

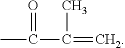

In particular, the compound may have a structure as follows:

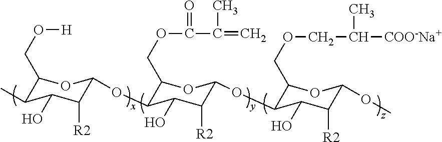

wherein:
x, y, and z are integers and each x, y or z is ≧1; and
each R₂ is the same or different, selected from the group consisting of:

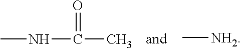

The compound may have an average molecular weight from about 10000 Da to about 15000 Da. The compound may have a degree of N-acetylation equal to or greater than 50%. For example, the degree of N-acetylation may be about 60%, 70%, 75%, 80%, 85%, 90%, 95%, 98%, 100%. In particular, the degree of N-acetylation may be greater than or equal to 80%. Even more in particular, the degree of N-acetylation is 90%.

Even more in particular, the compound may have a structure as follows:

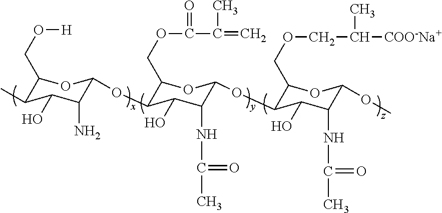

wherein x, y, and z are integers and each x, y or z is ≧1. The above structure is a non-limiting example. Accordingly, other structures may also be possible, depending on the degree of N-acetylation.

The hydrogel may be for use in several applications. For example, the hydrogel may be for use in biomedical applications. The biomedical applications may include, but is not limited to, any one of the following: drug delivery, wound dressing, bone substitute, bone cement composition, temporal joint spacers, dental cement composition, skin substitute, soft tissue distension, soft tissue repairing and/or replacement materials. In particular, the biomedical application is drug delivery.

The hydrogel according to any aspect of the present invention may further comprise at least one macromonomer. Any suitable macromonomer may be used. For example, the macromonomer may be as described above. In particular, the macromonomer is poly(ethylene glycol) diacrylate (PEGDA), copolymers thereof, or a combination thereof. The hydrogel according to any aspect of the present invention may further comprise at least one drug and/or at least one bioactive agent. Any suitable drug and/or bioactive agent may be used. For example, the bioactive agent may be as described above. Accordingly, the present invention also provides a hydrogel according to any aspect of the invention for use as a drug delivery agent and/or drug carrier.

Another aspect of the present invention is a composition comprising a compound according to any aspect of the invention having the following formula:

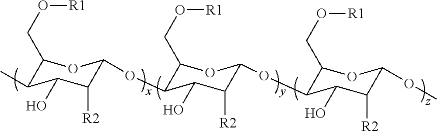

wherein:
x, y, and z are integers and each x, y or z is ≧1;
each R₁ is the same or different, selected from the group consisting of:

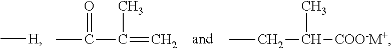

wherein M is a metal; and
each R₂ is the same or different, selected from the group consisting of:

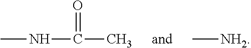

M may be any suitable metal. In particular, M is any monoelectropositive metal.

Even more in particular, M is sodium (Na) or potassium (K). In particular, at least one R₁ in the structure is:

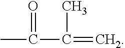

In an alternate embodiment, at least one R₁ in the compound comprises an unsaturated functional group. For example, the unsaturated functional group may be a functional group with a carbon-carbon double bond or a carbon-carbon triple bond. In particular, at least one R₁ in the compound comprises a carbon-carbon double bond. Even more in particular, at least one R₁ in the compound is:

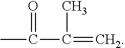

In particular, the compound may have a structure as follows:

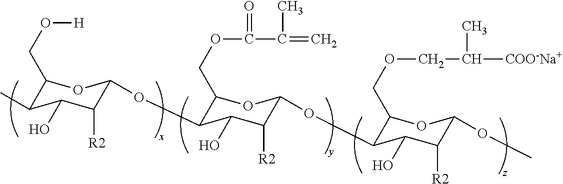

wherein:
x, y, and z are integers and each x, y or z is ≧1; and
each R₂ is the same or different, selected from the group consisting of:

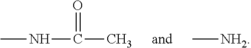

The compound may have an average molecular weight from about 10000 Da to about 15000 Da. The compound may have a degree of N-acetylation equal to or greater than 50%. For example, the degree of N-acetylation may be about 60%, 70%, 75%, 80%, 85%, 90%, 95%, 98%, 100%. In particular, the degree of N-acetylation may be greater than or equal to 80%. Even more in particular, the degree of N-acetylation is 90%.

Even more in particular, the compound may have a structure as follows:

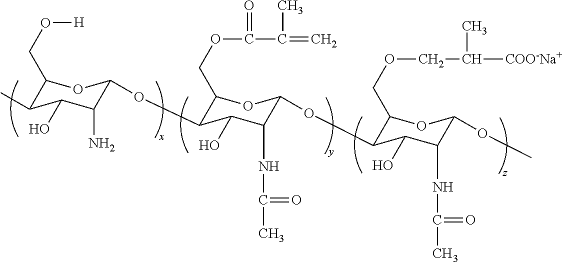

wherein x, y, and z are integers and each x, y or z is ≧1. The above structure is a non-limiting example. Accordingly, other structures may also be possible, depending on the degree of N-acetylation.

According to a particular aspect, the compound may be a hydrogel of the compound.

The composition may further comprise at least one macromonomer. Any suitable macromonomer may be used for the present invention. For example, the macromonomer may be as described above. In particular, the at least one macromonomer may be poly(ethylene glycol) diacrylate (PEGDA), copolymers thereof, and/or a combination thereof.

The composition may further comprise at least one solvent. The solvent may be any suitable solvent. The solvent may be as described above. In particular, the solvent may be water.

The composition may further comprise at least one osteoconductive material. Any suitable osteoconductive material may be used. The osteoconductive material may be as described above. In particular, the osteoconductive material may be as described above.

According to another particular aspect, the composition may further comprise at least one radiopaque material. The radiopaque material may be any suitable radiopaque material. In particular, the radiopaque material may be barium sulfate.

The composition may further comprise at least one polymerization initiator and/or at least one polymerization accelerator. Any suitable polymerization initiator and/or polymerization accelerator may be used. The polymerization initiator and/or polymerization accelerator may be as described above. In particular, the polymerization initiator may be ammonia persulfate (APS) and/or the polymerization accelerator may be N,N,N′,N′-Tetramethylethylenediamine (TEMED).

The composition may further comprise at least one polymerization inhibitor. The at least one polymerization inhibitor may prevent premature crosslinking of the composition. In particular, the polymerization inhibitor may prevent or reduce the premature generation of free radicals which lead to premature crosslinking. Such premature crosslinking may render the composition useless. Any suitable polymerization inhibitor may be used. For example, any suitable chemical or material which prevents free-radical polymerization may be used for the purposes of the present invention. The at least one polymerization inhibitor may delay or prevent crosslinking during storage, manufacture and/or transport of the composition. For example, the polymerization inhibitor may be hydroquinone, p-benzoquinone, trinitrobenzene, nitrobenzene or diphenylpicrylhydrazyl (DPPH). In particular, the polymerization inhibitor is hydroquinone. Only a small amount of polymerization inhibitor is required. For example, the polymerization inhibitor may be about 0.01-0.1 weight % based on the total weight of the composition.

The composition may also further comprise at least one bioactive agent. Any suitable bioactive agent may be used. For example, the bioactive agent may be as described above. In particular, the bioactive agent may be selected from the group consisting of: bone morphogenetic proteins (BMP), antibiotics, growth factors, therapeutic agents, and a combination thereof. Even more in particular, the bioactive agent may be vitamin B₁₂.

The composition according to any aspect of the present invention may be a bone cement composition and/or a dental cement composition. The composition according to any aspect of the invention may be used for several applications. For example, the composition may be used as bone void filler, bone cement, dental filler material, fixation cement for bone prosthesis implantation and/or filler of soft tissue spaces. According to a particular aspect, the composition may be for use in medicine.

The composition may be for use in orthopedic applications. For example, the composition may be for use in arthroplasty procedures of joints, for the fixation of prostheses to living bones. The composition may be for use in bone filling, bone repairing, bone implanting and/or for joining bone implants. The composition may be for use when reconstruction is necessary because of osteoarthritis, rheumatoid arthritis, traumatic arthritis, avascular necrosis, non-union of fractures of the neck of the femur, sickle cell anemia, osteoporosis, secondary severe joint destruction following trauma or other conditions (including fixation of some unstable fractures in metastatic malignancies), collagen disease and/or revision of previous arthroplasty procedures. The composition according to any aspect of the present invention may also be used for filling bone voids and/or gaps of the skeletal system, such as extremities, craniofacial, spine and pelvis, and for bone defects. The composition may also be used for distal radius void filling, tibial plateau void filling, tibial pilon void filling and/or calcaneal void filling.

The composition according to any aspect of the present invention may be generally used for fixation of artificial joints to bone stock. The composition may be used as fixation cement for bone prosthesis implantation. The composition may act more as a filler. For example, the composition may serve as a grout or interfacial material between the reamed medullary canal of the proximal femur or tibia for total hip or knee implants, respectively, and the metallic stem of the prosthesis. The composition applied to the medullary canal, for example, is intended to form a layer (mantle) of uniform thickness between the bone and the implant stem. This cement mantle is intended to mechanically interlock with the pores of the prepared bone and structurally compensate for the inability of the surgical technique to create a cavity in bone that exactly matches the shape of the total joint stem.

For the purposes of the present invention a “gap” refers to a cavity of the bone. The term “defect” refers to a condition which may be considered a disease and needs to be treated therapeutically, whilst with the term gap, it refers to a condition which is not necessarily a disease and may be treated non-therapeutically, for example for cosmetic purposes.

The composition according to any aspect of the present invention may also be for use in vertebroplasty and/or kyphoplasty. In particular, the composition may be used as a bone cement composition for vertebroplasty and/or kyphoplasty. Vertebroplasty and kyphoplasty are recently developed techniques for treating vertebral compression fractures. Percutaneous vertebroplasty was first reported by a French group in 1987 for the treatment of painful hemangiomas. In the 1990's, percutaneous vertebroplasty was extended to indications including osteoporotic vertebral compression fractures, traumatic compression fractures, and painful vertebral metastasis. In one percutaneous vertebroplasty technique, the composition may be percutaneously injected into a fractured vertebral body via a trocar and cannula system. The targeted vertebrae are identified under fluoroscopy. A needle is introduced into the vertebral body under fluoroscopic control to allow direct visualization. A transpedicular (through the pedicle of the vertebrae) approach is typically bilateral but can be done unilaterally. The material can also be delivered through an extrapedicular route.

Kyphoplasty is a modification of percutaneous vertebroplasty. Kyphoplasty involves a preliminary step that comprises the percutaneous placement of an inflatable balloon tamp in the vertebral body. Inflation of the balloon creates a cavity in the bone prior to composition injection. Further, the proponents of percutaneous kyphoplasty have suggested that high pressure balloon-tamp inflation can at least partially restore vertebral body height. In kyphoplasty, it has been proposed that the composition can be injected at lower pressures into the collapsed vertebra since a cavity exists to receive the composition—which is not the case in conventional vertebroplasty.

The composition according to any aspect of the invention may also be for use in dentistry and/or periodontal applications. Dentistry and/or periodontal applications may be for therapeutic dental procedures or non-therapeutic dental procedures. For example, non-therapeutic procedures may be cosmetic dental procedures. The composition according to any aspect of the present invention may be used for several clinical dental procedures including, but not limited to, pulp capping, management of root fracture, root perforation repair, root-end filling, barrier cement for intracoronal bleaching and/or apical barrier/plug. The composition may also be used as a temporary filling material to be placed in the tooth until a permanent restoration can be placed. The composition may also be used as a permanent filling material. The composition may further be used as a protective cement barrier in internal bleaching. The composition may be placed above the root canal filling material to minimise the leakage of bleaching agents through the root. Yet another use of the composition may be as a management of root fracture, in which the composition may be used to root fill a tooth with root fracture.

When the composition is used in pulp capping, it is used in the management of deep carious lesions, accidental mechanical pulp exposure and pulp exposure following traumatic accidents. The composition is placed over the exposed pulp and a restorative filling is then placed over it.

In the case of root perforation repair, this is carried out via an intracanal approach or using a surgical approach. The intracanal approach involves placement of the composition where the perforation is, in order to seal the perforation. The surgical approach is used to achieve the same after the intracanal approach fails or if the perforation is inaccessible through the access cavity of the tooth.

For root-end filling, it is a surgical procedure carried out to establish a seal at the root-end of the tooth. A cavity is prepared at the root end of the tooth and the composition is placed in the prepared cavity.

Apical barrier/plug is carried out in non-vital, immature permanent teeth with an open root apex. The composition is placed at the root end to create an apical plug which prevents the extrusion of root canal filling material.

Details of the procedures mentioned above may be found in any standard endodontics textbook (Pathways of the pulp. Eds Cohen S, Burns R C. 8^{th} Ed. Mosby Inc. St. Louis). Further, it would be known to a person skilled in the art how to perform the procedures.

Another aspect of the present invention is a method of preparing the compound having the following formula:

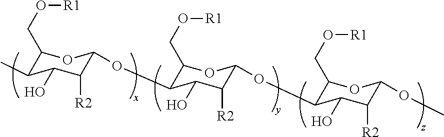

wherein:
x, y, and z are integers and each x, y or z is ≧1;
each R₁ is the same or different, selected from the group consisting of:

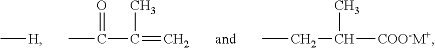

wherein M is a metal; and
each R₂ is the same or different, selected from the group consisting of:

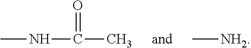

M may be any suitable metal. In particular, M is any monoelectropositive metal.

Even more in particular, M is sodium (Na) or potassium (K). In particular, at least one R₁ in the structure is:

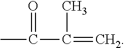

In an alternate embodiment, at least one R₁ in the compound comprises an unsaturated functional group. For example, the unsaturated functional group may be a functional group with a carbon-carbon double bond or a carbon-carbon triple bond. In particular, at least one R₁ in the compound comprises a carbon-carbon double bond. Even more in particular, at least one R₁ in the compound is:

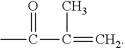

In particular, the compound prepared by the method may have a structure as follows:

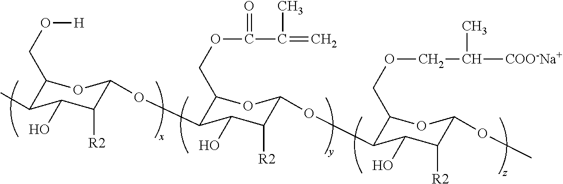

wherein:
x, y, and z are integers and each x, y or z is ≧1; and
each R₂ is the same or different, selected from the group consisting of:

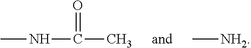

The compound may have an average molecular weight from about 10000 Da to about 15000 Da. The compound may have a degree of N-acetylation equal to or greater than 50%. For example, the degree of N-acetylation may be about 60%, 70%, 75%, 80%, 85%, 90%, 95%, 98%, 100%. In particular, the degree of N-acetylation may be greater than or equal to 80%. Even more in particular, the degree of N-acetylation is 90%.

Even more in particular, the compound may have a structure as follows:

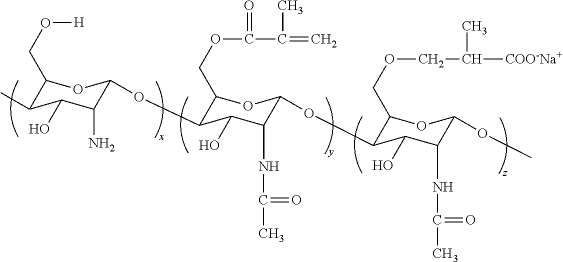

wherein x, y, and z are integers and each x, y or z is ≧1. The above structure is a non-limiting example. Accordingly, other structures may also be possible, depending on the degree of N-acetylation.

The method comprises the steps of:
(a) depolymerization of chitin; and
(b) esterification of the product of step (a) with methacrylic acid.

The step of (a) depolymerization of chitin may be achieved by using suitable acid and/or enzymes. For example, the depolymerization step may be achieved by subjecting chitin to hydrochloric acid for a suitable period of time. In particular, the chitin may be subjected to 10M hydrochloric acid for about 15 minutes. Suitable enzymes which may be used for the depolymerization step include, but are not limited to, chitinases, glucosaminidases, lysozymes and hydrolases.

The step of (b) esterification comprises chemical derivatization of chitin. In particular, the esterification step introduces R₁ into the compound to form chitin methacrylate.

The chitin may have the following formula:

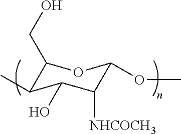

wherein n is an integer and n≧1.

According to a particular aspect, the esterification step (b) may be carried out in a lithium chloride/dimethylacetamide (LiCl/DMAc) solvent system.

Having now generally described the invention, the same will be more readily understood through reference to the following examples which are provided by way of illustration, and are not intended to be limiting of the present invention.

### EXAMPLES

Chitin was purchased from Eland Corporation Ltd, Thailand. The molecular weight of the chitin was approximately 200000 Da: Anhydrous N,N′-dimethylacetamide (DMAc) and poly(ethylene glycol) diacrylate (PEGDA, Mn=575) was purchased from Sigma-Aldrich. Methacrylic acid, N,N′-dicyclocarbodiimide (DCC), methacrylic acid and 4-dimethylaminopyridine (DMAP) were purchased from Fluka. LiCl was obtained from J. T. Baker. Vitamin B₁₂ and Hydrochloric acid (HCl) was bought from Merck, Germany. Photoinitiator Irgacure 2959 from Ciba Specialty Chemicals, Singapore. Other solvents used were technical grade without further purification.

### Example 1

### a) Preparation of Low Molecular Weight Chitin (LMW Chitin)

4.0 g of purified, chitin powder was placed in a 250 mL round-bottom flask (RBF) containing 120 mL of concentrated HCl solution (37%). The RBF was placed in a water-bath at 40° C. and stirring commenced to initiate hydrolysis. After 10 min, the flask was transferred into an ice-bath for cooling down and then the solution was poured into a 600 mL beaker. The hydrolyzed chitin solution was neutralised with 33.3% (w/w) NaOH solution in the ice-bath under magnetic stirring yielding a white precipitate that concomitantly formed a milky solution. The solution was centrifuged, the residue collected and dialyzed against water for 2 days, and lyophilized to obtain hydrolyzed chitin powder.

### b) Synthesis of Low Molecular Weight Chitin Methacrylate (LCMA)

Esterification of the carboxylic group and hydroxyl group in the presence of N,N-dicyclohexylcarbodiimide and 4-dimethylaminoamine is a traditional method that can be conducted under mild reaction conditions compared to the acylation of acyl halide and alcohol or esterification of carboxylic acid and alcohol in the presence of concentrated sulfuric acid. The reaction pathway of the esterification of chitin and methacrylic acid in 5% LiCl/DMAc solvent system is as shown:

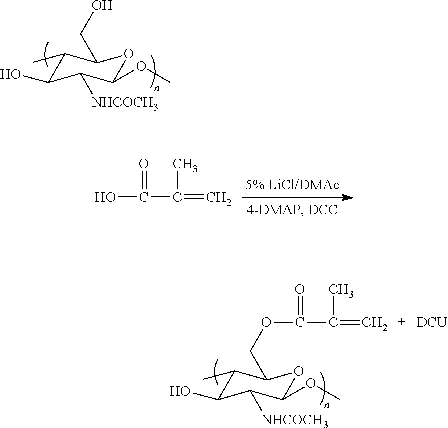

0.8 g of low molecular weight (LMW) chitin of molecular weight of approximately 10000 Da was dissolved in 20 mL of 0.5% LiCl/DMAc to make 4.0% (w/v) chitin solution in a 100 mL round-bottom flask. 2.0 mL (0.0236 mol, 6:1 as molar ratio based on pyranose of chitin) of methacrylic acid was dropped into the chitin solution using a syringe. 0.196 g (40% mol of molar of pyranose of chitin) of 4-DMAP (dissolved in 4 mL of anhydrous DMAc) and 4.88 g of DCC (dissolved in 4 mL of anhydrous DMAc, having the same molarity as methacrylic acid) was dropped into chitin solution in sequence. After stirring the mixture for 48 h at room temperature, a white precipitate was filtered off, which was identified as dicyclohexylurea (DCU), a by-product of DCC. Acetone was added to the filtrate to precipitate the product out. The product was washed with 50 mL acetone three times. The chitin derivative was dissolved in 50 mL of deionised water, subjected to dialyzing against deionized water at 4° C. for 5 days to remove water-soluble small molecules, and finally, the solution was lyophilized to obtain fluffy product of chitin methacrylate.

### Example 2

### Preparation of Composition, Formulation 1

0.2 g of chitin methacrylate as prepared in Example 1 was dissolved in a solution of 2.0 g of ultra-pure water and 3.0 g of poly(ethylene glycol) diacrylate with continuous stirring to make a viscous gel-like solution. A mixture of 3.2 g of hydroxyapatite and 0.8 g of barium sulfate was prepared and added to the viscous solution to make a fine paste without until no obvious granules were observed. 100 μl of ammonia persulfate (APS) (150 mg in 1 ml H₂O) was dropped into the paste and the mixture was stirred vigorously for 45 seconds. Thereafter, 50 μl of N,N,N′,N′-Tetramethylethylenediamine (TEMED) (200 μl in 1 ml H₂O) was added to the mixture. The paste was then ready for injection.

### Example 3

### Preparation of Composition, Formulation 2

0.2 g of chitin methacrylate as prepared in Example 1 was dissolved in a solution of 2.0 g of ultra-pure water and 3.0 g of poly(ethylene glycol) diacrylate with continuous stirring to obtain a viscous gel-like solution. A mixture of 3.6 g of hydroxyapatite and 0.4 g of barium sulfate was prepared and added to the viscous solution to make a fine paste until no obvious granules were observed. 100 μl of APS (150 mg in 1 ml H₂O) was dropped into the paste with vigorously stirring for 45 seconds, followed by the addition of 50 μl of TEMED (200 μl in 1 ml H₂O). The paste was ready for injection.

For the present formulation, the ratio of hydroxyapatite to barium sulphate was changed as compared to that in formulation 1 to study the difference in visualisation of cement under x-ray as shown in FIG. 4.

### Example 4

### Observation of Setting Time and Maximum Setting Temperature

The setting time and maximum setting temperature was performed at three different temperatures of the water bath: 25° C., 30° C., and 37° C. The composition was prepared according to the method described in Example 2, except volumes of APS and TEMED were varied as shown in Table 1. The setting time and maximum setting temperature were noted.

The paste of composition prepared according to the method of Example 2 was loaded in a 10 mL plastic single-use syringe and injected into the finger part of a latex glove. A thermocouple (Type T, 219-4680, RS Component Pte Ltd, UK) was inserted into approximately the middle of the paste and sealed by a copper wire. The sealed finger part of the glove was inserted into a water bath at pre-determined temperatures, each of 25° C., 30° C., and 37° C., to monitor how the temperature changed with time using the VirtualBench-Logger software (National Instruments, US). It generally took about 3 minutes for mixing, loading the paste and sealing the glove before starting to record the change in temperature.

TABLE 1
Setting time (tₛₑₜ) and maximaum temperature (Tₘₐₓ) of
composition prepared according to the method described in
Example 2 at 25° C., 30° C., and 37° C. with varying amounts of
polymerization initiator (APS) and polymerization accelerator (TEMED).

| Tempera- | Volume | Amount of | Setting | Max. Solid | |
|---|---|---|---|---|---|
| ture (Water | of APS | TEMED | Time | temperature | |
| Bath) | (μL) | (μL) | (min) | (° C.) | Remarks |
| 25 | 100 | 25 | 15.2 ± 0.9 | 28.5 ± 1.6 | Partially |
| | | | | | solidified |
| | 100 | 50 | 5.1 ± 0.2 | 33.6 ± 1.2 | Solidified |
| | 50 | 50 | — | — | Not |
| | | | | | solidified |
| 30 | 100 | 25 | 14.3 ± 1.1 | 35.6 ± 1.3 | Partially |
| | | | | | solidified |
| | 100 | 50 | 6.9 ± 0.8 | 46.2 ± 0.6 | Solidified |
| | 50 | 50 | — | — | Not |
| | | | | | solidified |
| 37 | 100 | 25 | 6.5 ± 0.2 | 51.6 ± 2.3 | Solidified |
| | 100 | 50 | 4.2 ± 0.4 | 58.6 ± 4.7 | Solidified |
| | 50 | 50 | 17.0 ± 1.6 | 42.4 ± 1.6 | Partially |
| | | | | | solidified |

The method of calculating the setting time and setting solidification temperature was in accordance with ISO 5833: Implants for surgery—acrylic resin cement. The setting temperature (Tₛₑₜ) is defined as:

$${T}_{set} = \frac{{T}_{max} + {T}_{wb}}{2} ,$$

where Tₘₐₓ is the maximum temperature during setting; and
T_{wb} is the temperature of the water bath.

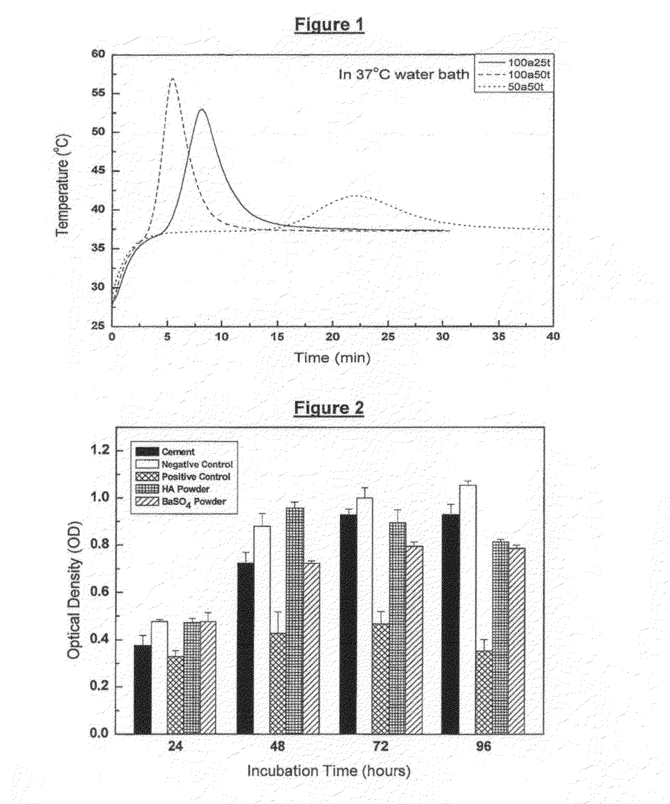

The setting time (tₛₑₜ) is the X-coordinate at the point of Tₛₑₜ on the dynamic exothermal curve as shown in FIG. 1 (for 37° C.). The exothermal curves for 25° C. and 30° C. are not shown.

### Example 5

### Toxicity of Composition

In vitro cytotoxicity of the composition prepared according to the method of Example 2 was evaluated by both direct contact assay and indirect contact assay in compliance with ISO-10993-5 (Biological evaluation of medical devices—Part 5: tests for in vitro cytotoxicity).

The cell line used was human osteoblast-like cells MG-63 from ATCC, USA. The cells were cultured in Minimal Essential Medium (MEM, Gibco BRL) containing 10% heat-inactivated fetal bovine serum (Gibco, BRL), 0.1% non-essential amino acids (Sigma), 1.5 g/L sodium bicarbonate (Cell Culture Grade, US Biological), 0.1 g/L penicillin (Sigma) and 0.1 g/L streptomycin (Sigma). For the direct contact assay, the paste was cut into a piece of 5 mm (length)×5 mm (width)×1 mm (height) and sterilized by γ-irradiation for 2 days at 2.5 Mrad. The paste was placed onto a confluent layer of cells and incubated for 96 hours. Samples were evaluated at 24, 48, 72 and 96 hours respectively. Polystyrene film and rubber latex glove were used as the negative and positive controls, respectively.

The indirect contact assay was performed by using the paste extract to contact the cells. The paste extract was prepared by soaking the paste in freshly prepared culture medium of composition as described above for 2 days and subsequently filtered through a 0.22 mm syringe filter to sterilize the mixture. The extract was exposed to a layer of cells and incubated for 96 hours, with samples being extracted at 24, 48, and 96 hours. Fresh culture medium and 0.5% phenol solution served as the negative and positive controls, respectively.

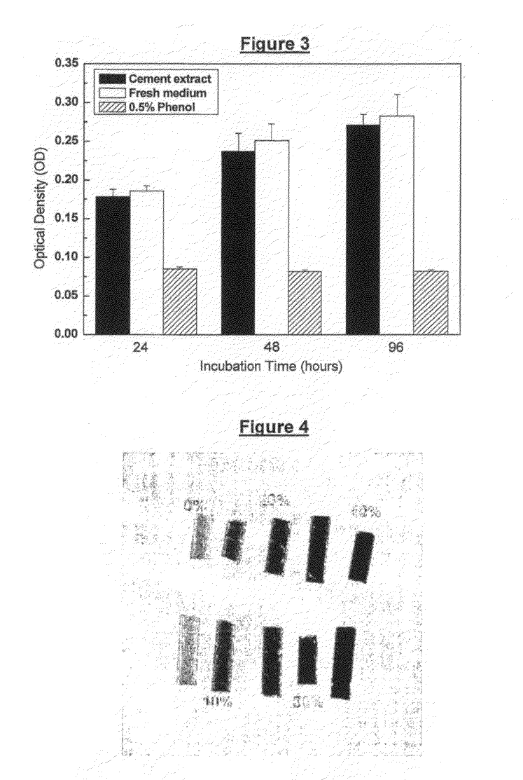

The optical density of each sample obtained from the direct contact assay and the extract assay are shown in FIGS. 2 and 3 respectively. The results were found to be comparable to the negative control, which means the composition itself is non-toxic to the selected cell lines and its extract also exhibits no cytotoxicity to osteoblast-like cells. In particular, the higher the optical density, the higher the cell proliferation.

### Example 6

### Visualisation Ability

The visualizing ability of the paste was evaluated by comparing five different formulations of the paste. In each formulation, the amount of chitin methacrylate and poly(ethylene glycol) diacrylate was the same as that used in Example 2. The total mass of the hydroxyapatite and barium sulfate was fixed, but in each of the five formulations, the ratio of hydroxyapatite to barium sulfate was altered, i.e. 100:0, 90:10, 80:20, 70:30, and 60:40 respectively. The cement is made to a cylindrical shape by injecting the paste into a glass tube and allowing the paste to solidify. The X-ray images obtained from OEC 6800 mini (Imaging3, Inc.) of each of the solidified paste formulations are shown in FIG. 4.

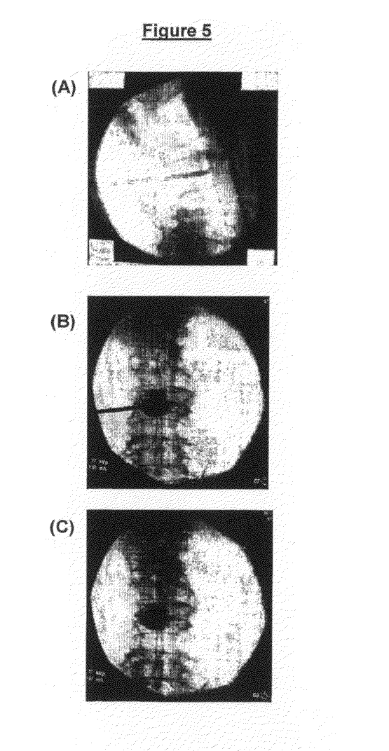

The paste formulation prepared according to Example 2 was injected into the spine of cadaver under fluoroscope monitoring to simulate the environment of the cement in animal or human spine. FIG. 5 shows the representative pictures that were taken at three stages (A) before paste injection, (B) during paste injection and (C) after the removal of the needle. The pictures show that there is enough contrast to guide a surgeon where he is introducing the paste into the bone site.

### Example 7

### Biological Performance in a Comparative Science Model

In vivo preliminary performance of the composition in a biological system was conducted using four pigs. Studies requiring the use of animal models were initiated after the research team was satisfied that the results of a comprehensive program to assess the bone cement performance and safety in vitro were very promising and warranted confirmation using an animal model. The composition was injected into the left and right distal femur. The four pigs were separated into two groups to host the composition for two different durations. One group was to host the composition for 1 week, and the other, for 5 weeks.

All surgical instruments and cement components were sterilized by autoclave or γ-irradiation. Four male pigs aged 7 months and weighing between 58-65 kg were used for the performance study of the bone cement. All animals were fasted overnight prior to the surgical procedure. Each animal was chemically restrained with intramuscular injection of ketamine (15 mg/kg), and its anesthesia state was maintained by 2.5% isoflurane inhalation through an anesthesia machine (Drager, Germany) via a 7 mm diameter endotracheal tube. The anesthesia machine was set in the self-respiration mode and the animal was monitored for SpO₂ status. The animal was shaved on the hind limbs and swabbed clean with 1% centrimide, followed by 0.05% chlorohexidine and finally with 1% povidine iodine before sterile draping for the surgical procedure.

A small cavity was laterally created with a surgical spatula in both the right and left distal femurs by removing cancellous bone. The mixed paste formulation was loaded into a 10 ml syringe and introduced into the cavity through a cement filler nozzle (Kyphon, USA) under fluoroscopic guidance. The animal was treated with antibiotic to reduce the risk of perioperative infection. Each pig was given oxytetracycline (7 mg/kg) by intramuscular injection. All four pig models survived after the surgery.

Postoperatively, each animal was maintained on oxytetracycline daily for 5 days and analgesic caprofen (2 mg/kg) daily for 3 days. The animals were hosted for 1 and 5 weeks before being sacrificed.

At sacrifice, the bones where the cement was injected was isolated for histological study. Each femur was stripped all of its soft tissues and fixed in ‘20 volume’ of 10% phosphate buffered formalin for 2 days. The bone and cement of interested was sawed off (i.e. the bone cavity filled with cement and enclosed by cancellous bone) and decalcified in rapid decalcifying agent (RDA) for 1 day. The decalcified tissue was dehydrated by processing with alcohol in the ascending concentrations of 70%, 95% and 100%. The dehydrated sample was embedded in paraffin wax to generate a tissue block and sliced using a microtome to give the histological sections of 5 μm thickness. The sections were permitted to adhere onto glass slides and dried in an 80° C. oven for 1 day. The sections were de-waxed with xylene and alcohol solutions, washed with water, and subsequently stained with haematoxylin and eosin (H & E) solution. The stained sections were washed and dehydrated with gradually increasing concentrations of alcohol and finally mounted in depax and glass cover for histological observation under light microscopy.

The results showed that there was no cutaneous erythema or discharge, good healing was shown and preservation of the skin, subcutaneous tissue and muscles around the tibia were earmarked for histology. There was no collection of exudates or tissue necrosis noted. These findings show that the composition is biologically acceptable to biological systems.

### Discussion of Examples 1 to 7

The results obtained from Examples 1 to 7 show that the formulation using the stated quantities provided the optimum that fulfils the requirements of a composition as follows:

(a) in terms of surgical procedure: being injectable, easy to handle, radiopacity, desirable viscosity and setting time of the composition;
(b) in terms of a subject's response to the composition: the subject's body's response to foreign implants vis a vis curing temperature, bioactivity, biologically compatible, degradation rate; and
(c) in terms of cost: cost of the components of the composition.

The most desired properties as primary considerations for a suitable composition for use as bone cement are injectability, a longer handling time of the cement ex-vivo, a short hardening time when injected into the subject's body and better compatibility to bone than the PMMA bone cement.

### Example 8

### 8.1 Preparation of LCMA Hydrogel by UV-Light The hydrogel was synthesized by dissolving the desired amount of LCMA (as prepared in Example 1) and Irgacure 2959 (5% w/w based on LCMA amount) in D.I. water together to make a homogenous precursor solution and subsequently placed under UV-light (Vilber Lourmat, France) at 365 nm to polymerize for 1 h. The solution was able to convert to hydrogel within the first 10-15 min but the exposure time was prolonged to increase the crosslinking degree.

### 8.2 Determination of Molecular Weight Distribution of Hydrolyzed Chitin by GPC

The molecular weight distribution of hydrolyzed chitin was performed using a Waters Gel Permeation Chromatography (GPC) system equipped with Waters LC (model 515) pump together with Waters 410 differential refractometer as a detector. Pullulan standard P-82 (Shodex, Japan) was used for plotting the standard calibration curve. The LMW chitin sample was dissolved in 5% LiCl/DMAc to make 2% (wt/v) chitin solution and eluted with 5% LiCl/DMAc at 0.8 mL/min through three column sets (Phenomenex 10 Linear, 300 mm×7.8 mm×10 μm) and a guard column at 65° C. The detector temperature was 40° C.

### 8.3 FTIR Characterization

The structure of LMW chitin was confirmed by FTIR, and the N-deacetylation degree of hydrolyzed chitin was evaluated as well. A small amount of LMW chitin methacrylate or hydrolyzed chitin sample was mixed well with fine KBr powder, ground and then made into pellets to be scanned on FTIR spectrometer (Bio-Rad Laboratories, Cambridge, Mass., USA) for 32 times at a resolution of 4 cm⁻¹.

### 8.4 Liquid Nuclear Magnetic Resonance (NMR)

¹H-NMR and ¹³C-NMR were used to determine the chemical structure of chitin methacrylate. A sample was dissolved in D₂O and recorded by Bruker ACF 300 NMR machine (300 MHz). Proton NMR was scanned 32 times while carbon NMR was scanned overnight.

### 8.5 Thermo Gravimetric Analysis (TGA)

Thermo gravimetric analysis was used to investigate the decomposition temperatures of LMW chitin and LCMA, as well as for the comparison to original chitin from shrimp shells. The dried samples (approximately 10 mg) (the samples were dried either by oven (for chitin) or freeze drying (for LCMA)) were placed in alumina pans, and heated from ambient temperature to 700° C. at 10° C./min under nitrogen gas with a flow rate of 100 mL/min in the TGA instrument (TGA 2960, TA Instruments, Inc). The data was analyzed using Universal Analysis Software (TA Instruments, Inc).

### 8.6 Powder X-ray Diffraction (XRD)

Wide angle X-ray diffraction was utilized to determine the crystal structure of chitin, depolymerized chitin, and LCMA. The powder of each sample was pressed in the holder and run on X-ray diffractometer (Siemens D5005) from 2θ=5° C. to 40° C. at the scanning step of 0.01° C./second.

### 8.7 Enzymatic Degradation Study

In vitro enzymatic degradation of hydrogel was conducted by using various concentrations of lysozyme solution. The pre-weighed sample of xerogel was soaked in a beaker containing 2 mL of lysozyme solution at 37° C. for different time periods and then taken out and rinsed with D.I. water to remove lysozyme and air-dried until a constant weight was achieved. The degradation rate was expressed as:

$$Degradation rate ( \% ) = \frac{( {W}_{x} - {W}_{d} )}{{W}_{x}}$$

where Wₓ is the mass of starting xerogel; and W_{d} is the mass of dried sample after degradation. The experiments were performed at least thrice.

### 8.8 Cytotoxicity Evaluation of Hydrogel

### (a) Cell Culture

Three types of cell lines including NCTC clone 929 (mouse fibroblast, ATCC. CCL-1), IMR-90 (human lung fibroblast, ATCC CCL-186), and MG-63 (osteoblast from osteosarcoma, ATCC CRL-1427) were used to assess the cytotoxicity of hydrogel in vitro as well as the cells adhesive ability and their morphology on the hydrogel surface. All cells were grown in the Minimal Essential Medium (MEM, Gibco BRL) containing 10% heat-inactivated fetal bovine serum (Gibco BRL), 0.1% non-essential amino acids (Sigma), 1.5 g/L sodium bicarbonate (Cell Culture Grade, US Biological), 0.1 g/L penicillin (Sigma) and 0.1 g/L streptomycin (Sigma) at 37° C. in a 5% CO₂/95% H₂O humidified atmosphere.

The direct contact method and extract assay were complied with ISO 10993-5 (Part 5: tests for in vitro cytotoxicity) by exposing either the hydrogel surface or the extract to the cells and examined by MTT assay to determine its cytotoxicity. MTT assay is a rapid colorimetric method based on the cleavage of a yellow tetrazolium salt (3-{4,5-dimethylthiazol-2-yl}-2,5-diphenyl tetrazolium bromide) to purple formazan crystals by mitochondrial enzymes of metabolically active cells and dissolved in DMSO to quantify at wavelength of 590 nm (G Ciapetti et al, 1993).

### (b) Direct Contact Assay

The hydrogel was dehydrated at room temperature for 48 h and in absolute ethanol for 3 h prior to sterilizing overnight under UV lamp. The dehydrated hydrogel was soaked in the fresh culture medium for 24 h to reach swelling equilibrium to obtain medium conditioned hydrogel before experiment. Cells were seeded in the 24-well plate at 5×10⁴ per well and incubated for 24 h at 37° C. to allow the cells to adhere to the bottom of the well. The medium was removed and the preconditioned hydrogel was gently placed in the wells to touch the cells. Controls using polystyrene (negative control) and latex rubber (positive control) were carried out in the same manner. 1 mL of new medium was added into each well and incubated at 37° C. in 5% CO₂ humidified atmosphere for 1, 2, 3, 4 days for further cells viability examination. 200 μL of MTT solution (5 mg/mL in 1×PBS) was applied to each well after removing the culture medium at each time point and continued to incubate for another 3 h to let healthy cells reduce tetrazolium salt to purple crystals. MTT solution was aspirated and 300 μL of DMSO was introduced to the well to dissolve crystal to result in purple solution and 200 μL of each sample was taken for colorimetric assay. The results were expressed as the percentage of viable cells, with each experiment being performed at least with three replicates.

The percentage of cells viability was calculated as follows:

$$Percentage viability ( \% ) = \frac{( {OD}_{S} - {OD}_{Pavg} )}{( {OD}_{Navg} - {OD}_{Pavg} )} \times 100 \%$$

where ODₛ is the optical density of sample; OD_{Pavg} is the average optical density of the positive control; and OD_{Navg} is the average optical density of the negative control.

### (c) Extract Assay (Indirect Contact Assay)

The fresh hydrogel was cut to 1 cm (L)×1 cm (W)×1.5 mm (H) and tenderly washed with Milli-Q water twice and then immersed in a well containing 1 mL of culture medium for 48 hours at 37° C. The cells were seeded in the well at a lower density of about 2×10⁴ cells/well to incubate for 24 h before testing. The hydrogel extract was sterilized by 0.22 μm filter and the cell culture medium was substituted by the extract and sustained incubating for 24 h and 48 h before MTT assay. Fresh culture medium and 0.5% phenol solution were employed as negative and positive controls, respectively. The positive control including phenol solution and cells were placed in a separate plate to prevent the phenol vapour from contaminating the hydrogel extract or the medium of the negative control. The procedure of MTT assay for each group of cell viability was the same compared to the direct contact method of (b). Again, the results were expressed as a percentage of viable cells with at least three replicates.

### 8.9 Cells Morphology Observation

Scanning electronic microscopy (SEM) was used to observe the cells' morphology and their adhesive ability towards the hydrogels. 50 μL of cell suspension (1×10⁶ cells/mL) was dropped cautiously onto the surface of culture medium equilibrated hydrogel in a 24-well plate and incubated at 37° C. in 5% CO₂/95% H₂O humidified atmosphere for 2 h for cell attachment onto the hydrogel. Subsequently, another 1 mL of fresh medium was carefully added to the wells to cover the hydrogel surface and placed back into the incubator. After incubating for 2 days or 5 days, the individual hydrogel were taken out and washed twice with PBS and then fixed with 2.5% glutaraldehyde for 1 h. The cell-fixed hydrogel were dehydrated with gradual concentration ethanol at a series of 50%, 70%, 80% and 95% each for 5 min, and in 100% ethanol about 15 min. The hydrogel were then dried by critical point drying. The dried sample was sputtered with gold and observed under SEM to evaluate the cell morphology and its interaction with the material.

### 8.10 Results and Discussion

### (a) Preparation and Characterization of Low Molecular Weight Hydrolyzed Chitin

Chitin can be readily depolymerized to form chitin oligomer or monomer mixture of glucosamine and N-acetyl-glucosamine by either enzymatic or mineral acids hydrolysis. Hydrochloric acid showed effective hydrolysis of chitin in short time without significant deacetylation (J A Rupley, 1964). Depolymerization of chitin by HCl does not undergo side reaction like O-sulphation in terms of hydrolysis by H₂SO₄ (GAF Roberts, 1992). The solubility of low molecular weight chitin in DMAc/LiCl dramatically improved and results in its chemical modification, which is easier compared to high molecular weight chitin due to its viscosity being pretty low.

The results of molecular weight distribution, percentage yield and degree of N-acetylation of six hydrolyzed chitin samples are presented in Table 2.

TABLE 2
Molecular weight distribution, yield and D.A. of hydrolyzed chitin (n = 6)

| Weight M.W. | Number M.W. | | *Degree of |
|---|---|---|---|
| (Da) | (Da) | Yield (%) | N-acetylation (%) |
| 10009 ± 640 | 9054 ± 372 | 61.6 ± 7.4 | 88.0 ± 4.8 |

The degree of N-acetylation (DA) was calculated according to the following equation (J Brugnerotto et al, 2001):

$$\frac{{A}_{1320}}{{A}_{1420}} = 0.3822 + 0.03133 ( DA )$$

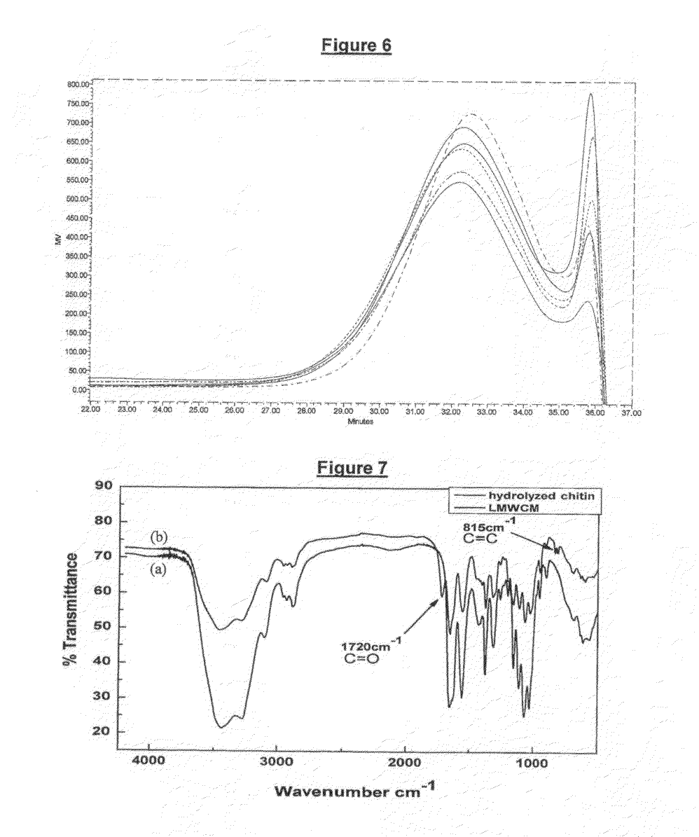

The molecular weight distribution of hydrolyzed chitin indicates that concentrated HCl could depolymerize starting from high molecular weight chitin (around 240 KDa by GPC) to obtain quite a narrow distribution and low molecular weight under the condition of 40° C. in a short period of time. The GPC profiles of six samples are presented in the FIG. 6 which indicated that the reproducibility was acceptable. Utilization of the dialysis membrane with 12000 molecular weight cut-off (MWCO) can contribute to get rid of the water-soluble chitin oligomers to obtain quite a narrow molecular weight distribution but at the expense of a low yield at mean time. The MWCO is an indication of which molecules will pass through the membrane. Therefore, a membrane with 12000 MWCO refers to a membrane which will allow molecules smaller than 12000 Da to pass through it.

### (b) Synthesis and Characterisation of Methacrylated Chitin and its Hydrogel

The photocrosslinkable chitin derivative was synthesized by reacting with methacrylic acid to introduce methacrylate onto chitin backbone using DCC as coupling agent. In particular, the chitin methacrylate had the following structure:

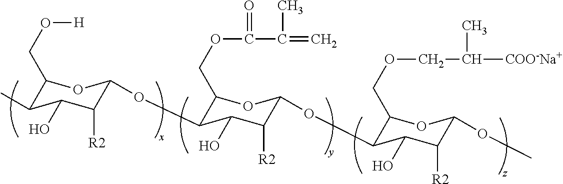

wherein:
x, y, and z are integers and each x, y or z is ≧1; and
each R₂ is the same or different, selected from the group consisting of:

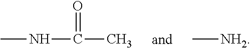

The compound had a degree of N-acetylation greater than or equal to 80%.

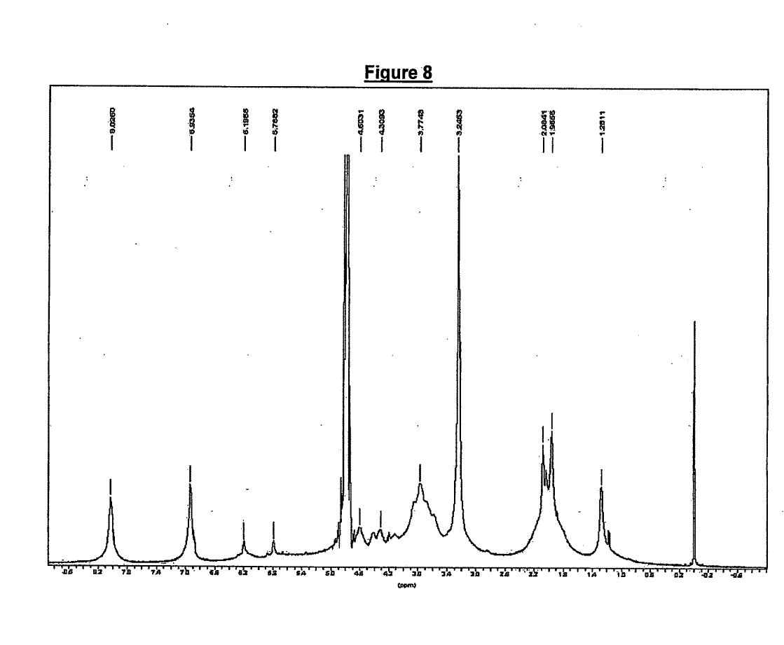

The structure was elucidated by FTIR and NMR spectrum as shown in FIGS. 7, 8 and 9. In the FTIR spectrum, the additional band at 1720 cm⁻¹ in methacrylated chitin spectrum was attributed to the stretching of the carbonyl group, and another band at 815 cm⁻¹ was ascribed to the pendant vinyl group of methacrylate group on chitin backbone (S H Kim and C C Chu, 2000). In the ¹H-NMR spectrum (FIG. 8) of chitin methacrylate, there were two peaks at 5.18 and 5.77 ppm which were corresponding to two protons of —C═CH₂ of the methacrylate group

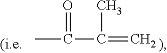

and the peak at 1.96 ppm was attributed to the —CH₃ on the methacrylate. Some chemical shifts belonging to chitin backbone could also be viewed, for example, 4.6 ppm was the proton at C1, the broad peaks between 3.4-4.2 ppm are the C2-C5 proton, and the typical chemical shift at 2.08 was attributed to —CH₃ on N-acetyl group of chitin. However, there were three additional peaks located at 8.21, 6.93 and 3.24 ppm which were assigned to DMAP. The presence of DMAP on the chitin backbone was rationalized in terms of the occurrence of a side reaction between the hydroxyl groups on chitin and methacrylic acid via Michael addition in the presence of DMAP as shown in the scheme below (Z B Zhang and C L Mccormick, 1997). The by-product with —COOH can further conjugate with DMAP base to form salt. The formation of a salt was also the possible reason for LCMA dissolving in water. This side reaction was confirmed by the additional chemical shift near 1.28 ppm corresponding to proton at position (a) of the product in the scheme shown.

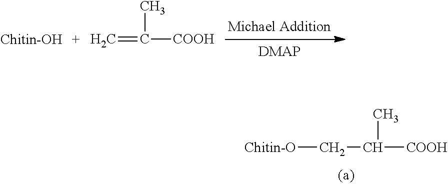

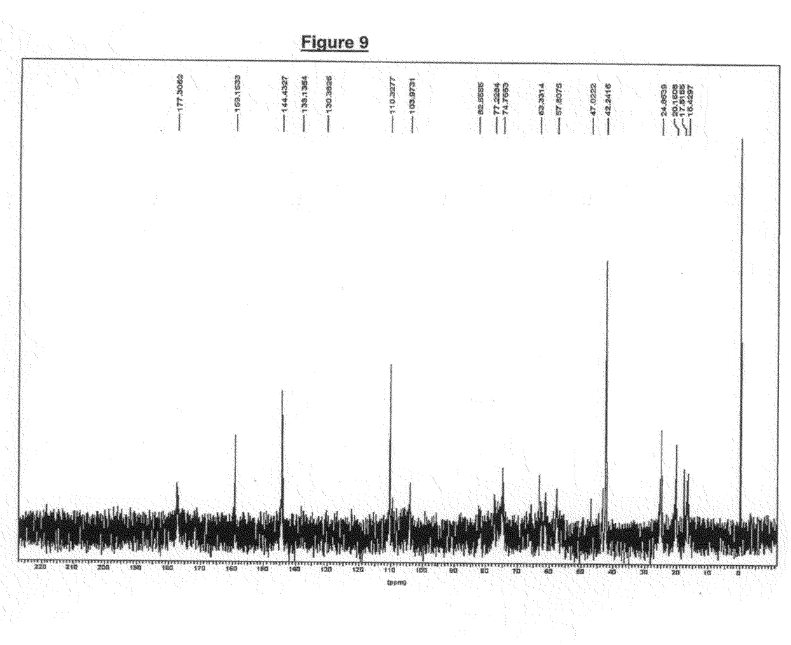

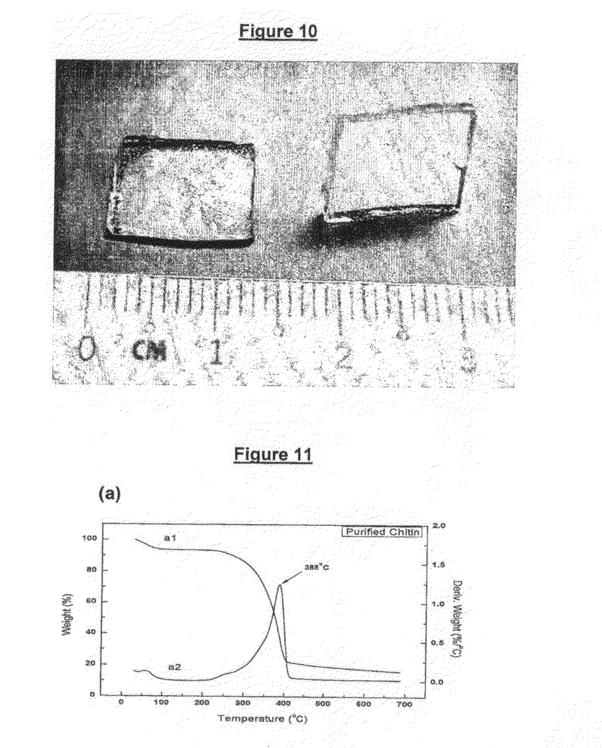

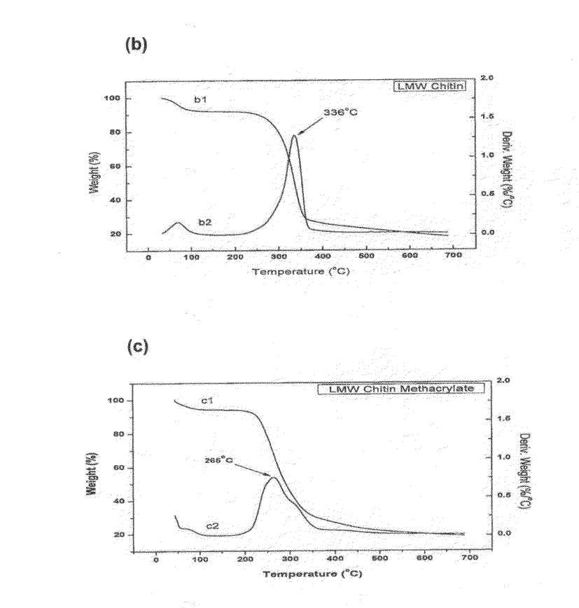

In the ¹³C-NMR spectrum shown in FIG. 9, two weak peaks at 130.3 and 138.1 ppm were assigned to two carbons at the unsaturated (—C(CH₃)═CH₂) of methacrylate group. There were two peaks near 20 ppm assigned to the methyl group on chitin's N-acetyl group (24 ppm, —NHCOCH₃) and methacrylate group (20 ppm, —C(CH₃)═CH₂). Another two peaks near 16 ppm were probably due to the carbons on the grafted group in the Michael addition by-product which are 17.5 ppm (—O—CH₂—CH(CH₃)—COOH) and 16.5 ppm (—O—CH₂—CH(CH₃)—COOH). The obvious strong chemical shifts at 42.2, 110.3, 144.4 and 159.1 ppm were assigned to DMAP. Other chemical shifts corresponded to chitin backbone could be found in the spectrum as described in Table 3:

TABLE 3
Chemical shifts of carbon belong to chitin backbone

| | C1 | C2 | C3 | C4 | C5 | C6 | NHCOCH₃ |
|---|---|---|---|---|---|---|---|
| ¹³C chemical | 103.9 | 57.8 | 74.7 | 82.5 | 77.2 | 63.6 | 177.3 |
| shifts | | | | | | | |

From the elucidation of the chemical structure of LCMA, it was concluded that the successful attachment of methacrylate group onto chitin was achieved. The degree of substitution of both methacrylate and the by-product were calculated according to the following:

The first step is to get the ratio of methacrylate chitin portion and side reaction product portion.

$$\frac{x}{y} = \frac{( {I}_{6.19} + {I}_{5.78} ) / 2}{( {I}_{8.21} + {I}_{6.93} ) / 4} \qquad Eq . 3.1$$

where:
x is the DA of methacrylate;
y is the percentage of DMAP-containing by-product on the polymer chain; and
each of I_{6.19}, I_{5.78}, I_{8.21} and I_{6.93} refers to the quantity of H¹ in peaks denoted as 6.19, 5.78, 8.21 and 6.93 respectively in FIG. 8.

In particular, the values of I_{6.19}, I_{5.78}, I_{8.21} and I_{6.93} refers to the value of the signal integration at the respective peaks shown in FIG. 8. The values can be obtained electronically from the NMR machine.

Hence, the ratio of methacrylate-pending chitin (x) and DMAP-pending chitin (y) can be obtained from Eq. 3.1 by integrating two protons on —C(CH₃)═CH₂ and four protons belonging to the pyridine ring of DMAP.

The degree of substitution of either methacrylate-pending chitin or DMAP-pending chitin can be further calculated by Eq. 3.2 as follows:

$$\frac{x / 2}{( DA / 3 ) + ( x / 3 ) + ( y / 3 )} = \frac{( {I}_{6.19} + {I}_{5.78} ) / 2}{( {I}_{1.96} + {I}_{2.08} ) / 3} \qquad Eq 3.2$$

where:
DA is the degree of N-acetylation of chitin for the reaction; and
each of I_{6.19}, I_{5.78}, I_{1.96} and I_{2.08} refers to the quantity of H¹ in peaks denoted as 6.19, 5.78, 1.96 and 2.08 respectively in FIG. 8.

In particular, I_{2.08} is the signal integration of —CH₃ on N-acetyl group of chitin and I_{1.95} is the integration of methyl of substituted groups either

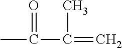

or

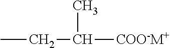

### in FIG. 8.

According to the above equation and the integration of H-NMR spectrum, the degree of substitution of methacrylate group is about 31.9%±1.8% by averaging four samples, which was equivalent to the group

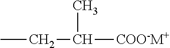

portion being approximately 29.8%±2.3%.

The solubility tests of LCMA was carried out in organic solvents or water, and the results showed that the product is capable of well dissolution in DMSO and DMAC/LiCl about 20% (w/v); in the case of pure water, its maximum concentration was not more than 7% (w/v) and formed a very viscous solution.

The above results made it possible to polymerize LCMA from its aqueous solution to form hydrogel under UV-irradiation with association of a photoinitiator. The LCMA aqueous solution converted to a transparent hydrogel as shown in FIG. 10 in about 10-15 min in the presence of a photoinitiator, whereas no hydrogel was observed without the presence of a photoinitiator by UV-irradiation even after a few hours.

### (c) Thermal Characteristics of LCMA

FIG. 11 shows the TGA patterns of purified chitin, LMW chitin, and LCMA. Weight loss curves of both chitin (a1) and LMW chitin (b1) have two decomposition stages: the first is around 50-100° C. due to the loss of residual water; the second stage is a little different between the two chitin thermograms, 240-410° C. for chitin and 220-370° C. for LMW chitin. The lower second decomposition stage in LMW chitin was attributed to the lower degree of polymerization in chitin after strong acid hydrolysis (SC Moldoveanu, 1998). However, three stages were found in the LCMA thermal analysis pattern (c1). The first stage from 50-100° C. was still due to the evaporation of water, the second and third stages overlay each other and their temperatures spanned from 190-380° C., suggesting that the sample consisted of LCMA and un-substituted chitin. Moreover, the onset temperatures of the three samples were also quite different. The temperatures were 315° C., 280° C. and 230° C. for chitin, LMW chitin and LCMA, respectively.

In the derivative weight loss curves of the three samples, the temperature of maximum weight loss (Tₘₐₓ) in the chitin curve (388° C., curve a2) was higher than the temperature of LMW chitin (336° C., curve b2) and LCMA (265° C., in curve c2). The derivative curve of LCMA (curve c2) was clearer than its weight loss curve to observe the heteropolymeric structure in the sample because there was a small peak on the right shoulder of main peak around its Tₘₐₓ which was ascribed to a much higher decomposition temperature of chitin. These results suggested that chemical modification of chitin results in lower thermal stability than the starting chitin material (S Tanodekaew et al, 2004). In addition, the low molecular weight chitin did not alter the thermal property of chitin too much.

### (d) Crystalline Analysis of LCMA by XRD

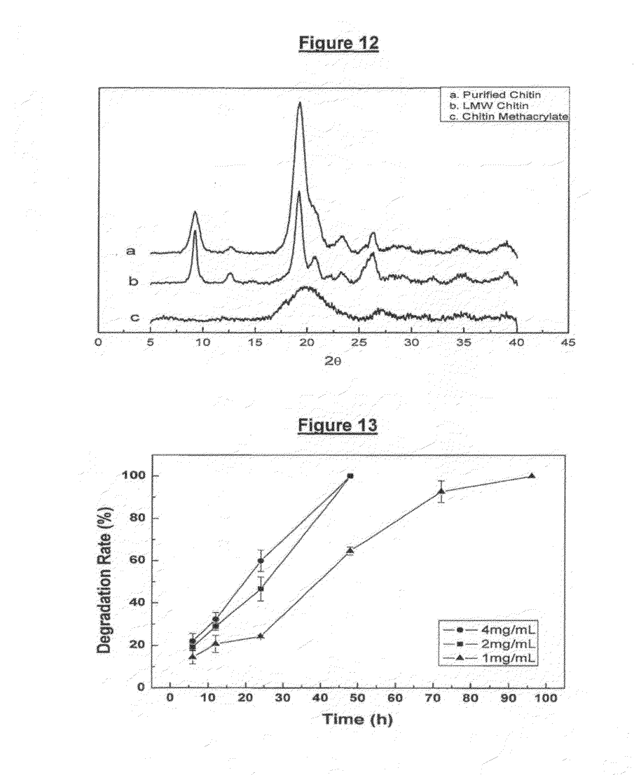

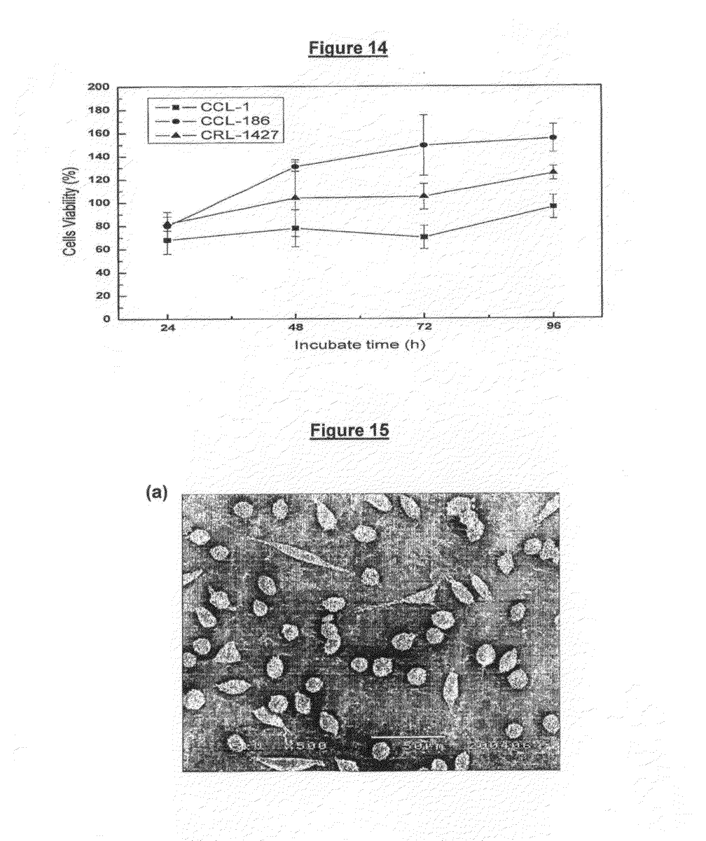

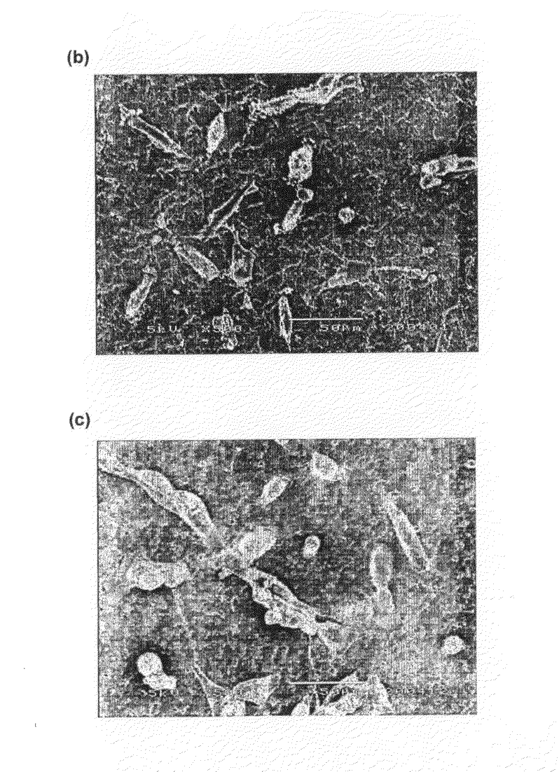

The XRD patterns of chitin, its LMW product and LCMA are presented in FIG. 12. Five main peaks could be observed in both chitin (a) and LMW chitin (b) patterns at 2θ=9.27, 12.66, 19.19, 23.20, and 26.31° in agreement with reports from literature (G Cardenas et al, 2004). The results indicate that chitin's crystalline structure remained essentially unchanged after acid hydrolysis. However, almost no crystal structure was found in the LCMA pattern (c). This indicates that after chemical modification of chitin, LCMA has an amorphous structure.

### (e) Degradation of Hydrogel In Vitro by Lysozyme

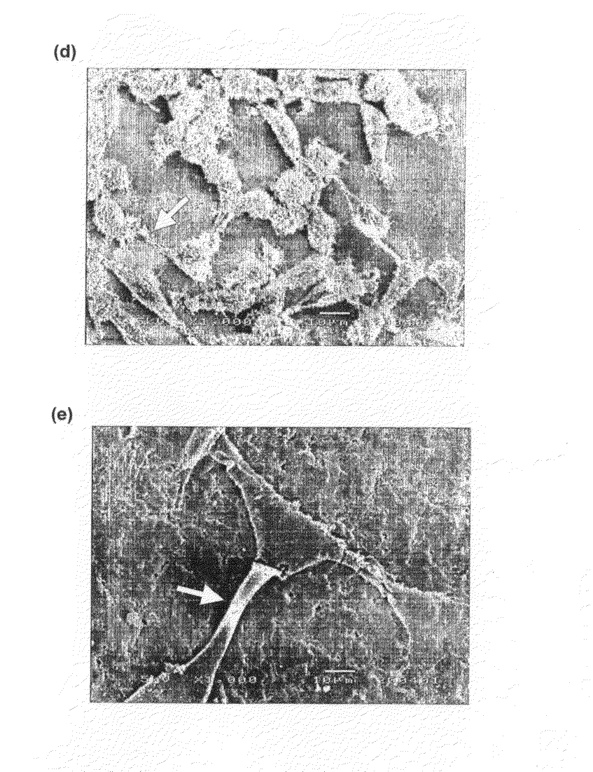

Chitin and its derivatives are well known to be readily to be depolymerized by lysozyme in its film or gel formation (K Tomihata and Y Ikada, 1997). The investigation of enzymatic degradation of hydrogel was carried out using different lysozyme concentrations in 0.01 M pH 7.0 PBS at 1.0 mg/mL, 2.0 mg/mL, and 4.0 mg/mL in 37° C. water bath. FIG. 13 shows the results of the degradation rate of hydrogel treaded by different lysozyme concentrations. The findings show that the photocrosslinked hydrogel can be totally degraded within 48 hours by 2 mg/mL or 4 mg/mL of lysozyme solution, while it takes a longer time up to 96 hours in the case of 1 mg/mL lysozyme solution to see complete dissolution of the hydrogel. Therefore, the amount of enzyme is critical to the entire degradation period of the hydrogel. In addition, the effective enzymatic degradation by lysozyme indicates that the contiguous N-acetyl groups on the unit of chitin, which is serving for the binding point with the active site of lysozyme (S H Pangburn et al, 1982), still exists in low molecular weight methacrylated chitin after acid hydrolysis and chemical modification. This is very imperative for the chitin based photocrosslinked hydrogel for use in biomedical applications.

### (f) In Vitro Cytotoxicity Evaluation

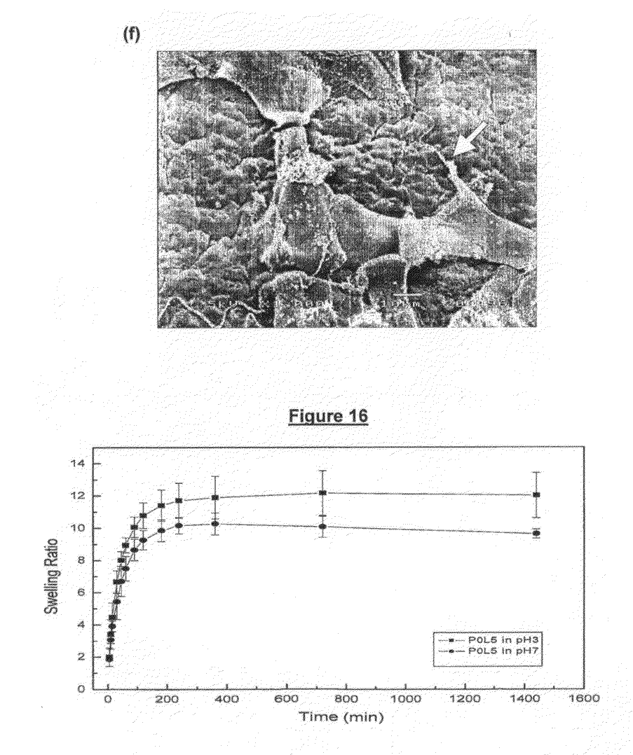

The in vitro cytotoxicity evaluation of biomedical materials is a necessary prelude to further in vivo study to establish the bioacceptability of the materials. Three types of cell lines were selected for assessing the cytotoxicity on the hydrogels directly or indirectly using its extract in culture medium. The percentage of cell viability refers to the positive and negative controls shown in FIG. 14 and Table 4. The results have been expressed as the mean of the percentage of viable cells with standard deviation. Each experiment was performed with at leak three replicates. The percentage of cell viability is calculated as indicated at Example 8.8 (b) above.

The findings for the direct contact method showed that the cell viabilities of the three cell lines are all approximately above 70% at 24 h, and increased to 150% for human fibroblast (CCL-186), 125% for osteoblast (CRL-1427) and 96% for mouse fibroblast (CCL-1) after 96 h incubation. The viability on the selected cell lines from human after 48 hours incubation increased to more than 100% probably due to the sizes of human fibroblast or osteoblast being quite large which caused it to attach on the surface of the hydrogel after it was covered by hydrogel, and subsequently proliferate on the specimen. The rougher surface of the hydrogel than polystyrene can stimulate the human osteoblast or fibroblast to be easier attaching and growing on the hydrogel which led to higher viability of the cells on the hydrogel (B D Boyan et al, 2001). This result is quite similar to the consequence in the report of Chow et al., 2002, that the cells viability of CCL-1 was also 20% lower than CCL-186 human fibroblast cell line.

The quantitative cytotoxicity examination on the hydrogel extract without dilution is shown in Table 4. The results showed that the extracts also expressed good viability in comparison with control after 48 hours of incubation. Two human cell lines have more than 90% viability compared to the control, and mouse cell lines are not less than 60% after 48 hours. Again, the viable ability of mouse fibroblast in hydrogel extract is slightly lower than human fibroblast or osteoblast. From the aspect of the cell viability either on hydrogel or its extract medium, the material can be regarded as being non-toxic to the human fibroblast or osteoblast, which leads to chitin-based hydrogel being useful in biomedical applications, for example as would healing material or as orthopedic material.

TABLE 4
Percentage of cell viability of extract assay.

| Incubation | Cells Viability (%) (Mean ± SD) | | |
|---|---|---|---|
| Time | CCL-1 | CCL-186 | CRL-1427 |
| 24 h | 78 ± 3 | 98 ± 7 | 77 ± 9 |
| 48 h | 62 ± 2 | 98 ± 3 | 92 ± 8 |

### (g) Cell Morphology on Hydrogel

SEM was used to observe adhesion, spread and proliferation of three types of cell lines on the surface of hydrogel after incubation in 37° C. for 2 or 5 days. The exemplified photographs, as shown in FIG. 15(a) to (c) on day 2 clearly show that all three kinds of cells were adhesive on the surface of hydrogels.

Interestingly, most of mouse fibroblasts (CCL-1) were still round-shaped and only a few cells formed a spindle-shape. On the contrary, both CCL-186 or CRL-1427 displayed most of cells with spindle-shape and were spread on the surface as well. The observation gives a clue that the mouse fibroblast is growing much slower and delayed its colonization compared to human fibroblast or osteoblast on hydrogel, and this also indicates that the quantitative cytotoxicity, both by direct contact method and extract assay, in terms of the percentage of cells viability is always lower in the case of CCL-1 mouse fibroblast than those data of human fibroblast and osteoblast. In fact, the morphology of mouse fibroblast on day 2 is very similar to the photomicrographs in the report by Muzzarelli et al., 2005, that only few fibroblasts showed spindle shape and started to spread on dibutyryl chitin film after incubation for two days. The photomicrographs of the cells-hydrogel surface on the fifth day (FIGS. 10 (d) to (f)) show that cells had spread on the material and proliferated. The fibrils of cells (indicated by the arrows) extended on the surfaces of the hydrogels or cells connected with each other demonstrate that the cells adhered very well. From the illustrated photomicrograph of cell morphology on the fifth day, it is obvious to distinguish that the sizes of mouse fibroblast in its spindle shape which are about 10 μm are smaller than in the corresponding state of human fibroblast or osteoblast which are approximately 50 μm. This shows that cell-hydrogel attachment and proliferation when conducting the direct contact assay to evaluate the cytotoxicity and results in predominantly higher cells viability of human cell lines than mouse fibroblast.

### (i) Conclusion

The low molecular weight chitin based derivative, chitin methacrylate, exhibits good solubility in water and DMSO, and can readily form hydrogel under ultraviolet light irradiation. Its good enzymatic degradation makes it possible for use as biodegradable drug delivery system. Hydrogel and its extract did not show remarkable toxicity to the human fibroblast and osteoblast as well as mouse fibroblast within the examined period. The observation of cell morphology and its interaction with hydrogel demonstrate that cells can attach fast and proliferate very well on the surface of hydrogel which make this photocrosslinked chitin-based hydrogel useful as tissue engineering scaffold.

### Example 9

### 9.1 Preparation of Hydrogel by Photocrosslinking of LCMA and PEGDA

Desired amounts of Irgacure 2959 was dissolved in water by gently stirring and heating to obtain a clear solution. The solution was then cooled down to room temperature. PEGDA was optionally added to the photoinitiator solution, followed by dissolving LCMA into the solution. The hydrogel precursor solution was poured into a plastic container and placed under a UV lamp (Vilber Lourmat, France) at 365 nm for about 30 min to allow polymerization to take place. The hydrogel was carefully washed with D.I. water twice for further study. The amounts of photoinitiator, PEGDA and LCMA are summarized in Table 5.

TABLE 5
Components of each hydrogel

| Samples ID | LCMA* | PEGDA* | Irgacure 2959** |
|---|---|---|---|
| P0L5 | 5% | 0% | 5% |
| P5L5 | 5% | 5% | 5% |
| P10L5 | 5% | 10% | 5% |

*The percentage of LCMA or PEGDA was based on the mass of water used.
**The percentage of Irgacure 2959 was based on the total mass of LCMA and PEGDA

### 9.2 Swelling Behavior of Hydrogel

The photocrosslinked LCMA hydrogel P0L5 obtained from Example 9.1 was cut into small disks and dried in vacuum for 2 days to obtain a constant mass. The xerogel was accurately pre-weighed before immersing in pH3.0 or pH7.0 phosphate buffer solution at 37° C. The swollen hydrogel was taken out of PBS at different time intervals and carefully blotted by wipers before being weighed. The total time for incubating the hydrogel in the PBS was 24 h because the weight of hydrogel did not increase anymore after that, which indicated that the water uptake-equilibrium of hydrogel had reached. The equilibrium water content (EWC) can be expressed as:

$$EWC ( \% ) = \frac{( {W}_{s} - {W}_{d} )}{{W}_{s}} \times 100$$

wherein Wₛ is the mass of the hydrogel in the equilibrium swollen state; and W_{d} is the mass of the xerogel.

The swelling ratio (SR) at each time interval was calculated by the following equation:

$$SR ( \% ) = \frac{( {W}_{t} - {W}_{d} )}{{W}_{d}} \times 100$$

wherein Wₜ denotes the mass of the hydrogel at different times t. Each experiment was performed in triplicate and the results were plotted as a mean value with standard deviation bar at each time interval. The swelling kinetics was investigated by the means of weighing the mass of hydrogel at each time interval.

### 9.3 Differential Scanning Calorimetry (DSC)

Differential scanning calorimetry was used to explore the water content of the hydrogels prepared in Example 9.1. The experiments were carried out using a 2920 DSC instrument and data was analyzed by Universal analysis (TA Instrument, Inc). A swollen hydrogel of known weight was sealed in an aluminium hermetic pan, and it was cooled in the instrument chamber to −15° C. by dry ice, then heated up to 15° C. at a rate of 1° C./min under nitrogen gas at flow rate of 50 mL/min.

In general, the water in the hydrogel consists in three forms: free water, freezing bound water, and non-freezing bound water. Free water is the water that does not participate in forming the hydrogen bonds with the polymer molecules. Freezing bound water is the water that has weak interactions with the polymer. Both free water and freezing bound water show endothermic peaks in the DSC curve, and the contents of these two kinds of water can be calculated by integrating the endothermic peak and dividing by the integration of the peak near 0° C. of pure water. However, non-freezing bound forms hydrogen bonds with the polymeric network. Hence, no endothermic peak can be observed in the DSC curve. The nonfreezing bound water content is conveniently obtained from the equation shown (S J Kim et al, 2004; M B Ahmad and M B Huglin, 1994; Y Liu and M B Huglin, 1995):

$${W}_{b} ( \% ) = {W}_{t} - ( {W}_{f} + {W}_{fb} ) = EWC - \frac{{Q}_{endo}}{{Q}_{f}} \times 100$$

W_{b} refers to the nonfreezing bound water, Wₜ refers to the total water in the swollen hydrogel which can be substituted by the EWC value, W_{f} is the free water and W_{fb} is the freezing bound water. W_{f} and W_{fb} can be approximately calculated by the integration of the endothermic peak. Q_{endo} and Q_{f} are the heats of fusion in the hydrogel and pure water, respectively, and they can be integrated by using the Universal Analysis software.

### 9.4 Scanning Electronic Microscopy (SEM)

Scanning electron microscopy was employed to observe the surface morphology of the synthesized LCMA and LCMA/PEGDA hydrogels. The hydrogels of three different components (P0L5, P5L5, P10L5) in the relaxed state were instantly cryofixed in the acetone and dry ice mixture and freeze dried for 3 days. The cryofixation greatly maintained the structure of the hydrogel after freeze drying in contrast with the common freezing procedure in the refrigerator because normal freezing process expands the volume of the substance during the transformation from water to ice. The dried polymer matrix was coated with gold for 45 s at 20 mA using an auto fine coater (JEOL, JFC-1600, Japan). The microphotographs were taken by scanning electron microscopy (JEOL, JSM-5200) with an accelerating voltage of 5 kV at various magnifications.

### 9.5 Release Profile of Vitamin B₁₂ from Hydrogel and Xerogel

Vitamin B₁₂ (VB₁₂, FW=1355.4, Merck) was selected as the model drug to study the release profile of hydrogels as potential drug delivery system. Each dehydrated hydrogel (P0L5, P5L5 and P10L5) was soaked in 5 mL of 10 mg/mL VB₁₂ solution for 48 h. The drug loading capacity was determined by the total amount of VB₁₂ released from the hydrogel into Milli-Q water. The value was expressed as loading capacity, determined according to the following equation:

$$Loading capacity ( \% ) = \frac{{W}_{VB 12}}{{W}_{drymass}} \times 100 \%$$

where W_{VB12} is the total mass of VB₁₂ in the hydrogel, determined by UV spectrometry and W_{dry mass} is the pre-weighed mass of xerogel.

The surface of the VB₁₂-saturated hydrogel was blotted with a red solution and directly used for the release profile study or dehydrated at room temperature until a constant weight was achieved to study the release properties of the xerogels. 0.1M HCl solution (to simulate gastric fluid) or Milli-Q water (to simulate intestinal conditions) was prepared as a release medium. The experimental procedure was similar to that described in T Coviello et al, 2005.

In each individual experiment, hydrogel or xerogel was placed in 150 mL of releasing medium in a conical flask at 37° C. with continuous shaking at 100 rpm in incubator (Sanyo, Japan). 5 mL of sample solution was aliquoted from the conical flask and replaced with another 5 mL of fresh releasing medium. The sample solution was diluted with 5 mL of Milli-Q water before measuring its absorbance at 361 nm. The concentration of released VB₁₂ at each time point was calculated directly by the UV spectrometer's software at its quantitative mode according to the regressive line as a function of concentration and absorbance of various standard known concentration VB₁₂ solutions. The concentration of standard VB₁₂ solution was 0.2, 0.4, 0.8, 1.2, and 1.6 mg/mL, respectively. The time intervals of the retrieved sample solutions were 2.5, 5, 10, 15, 30, 60, 120, 180, 240 minutes. Each experiment was conducted in triplicate.

### 9.6 Results and Discussion

### (a) Swelling Behavior of Hydrogel

(i) Swelling Ratio (SR)

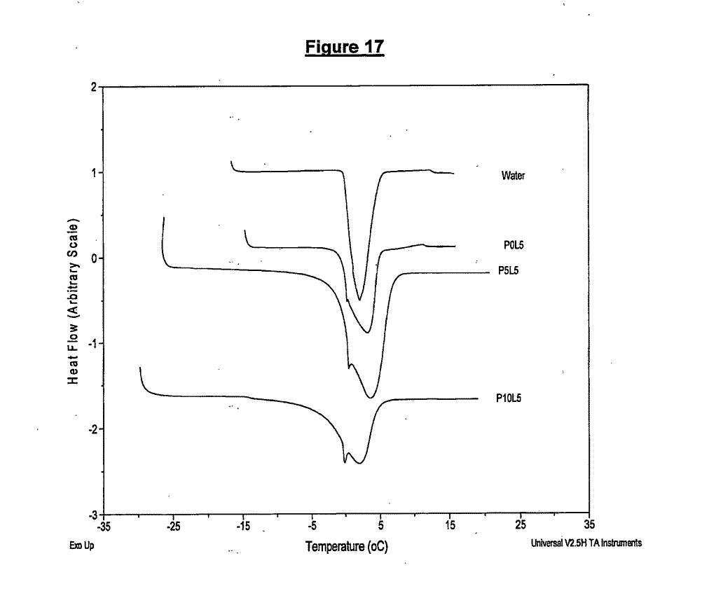

FIG. 16 shows the swelling ratio at different time intervals of P0L5 hydrogel in acidic (pH 3) and neutral (pH 7) solution. The final swelling ratio value was about 12 and 10 at pH 3 and pH 7 medium, respectively. The SR of hydrogel in the acidic solution was slightly higher than in the neutral solution because of the presence of a small amount of amino group in the chitin derivative as the starting hydrolyzed chitin was not fully N-acetylated. In acidic media, the amino group is protonated which induces the electrostatic repulsions between the polymer segments and exhibits the relaxation of the polymer network chains. However, such interactions will be reduced in the pH 7.0 PBS due to the lack of the ionization of the amino group (S N Khalid et al, 2002).

(ii) Swelling Kinetics

The investigation of the mature of water diffusion into network is important for the proposal of the controlled-release devices for drugs or bioactive agents. The typical description of transport mechanism of low molecular weight compounds through the polymeric network can be expressed as the following empirical equation (O Carmen et al, 1999; J Crank, 1978):

$$\frac{{M}_{t}}{{M}_{s}} = k {t}^{n}$$

where Mₜ is the mass of water of absorbed into the hydrogel at time t and M₈ is the amount of water in the equilibrium swelling state of hydrogel. k and n are constants. The diffusional exponent n can be calculated by the slope of the linear curve which is obtained by plotting log (Mₜ/M₈) as a function of log (t). In the case of n=0.5, the transporting mechanism indicates the Fickian diffusion, and when 0.5<n<1, it is called non-Fickian or anomalous diffusion. The values of n and k at the different pH level is shown in Table 6.

TABLE 6
Values of n, k of LCMA hydrogel in various pH buffer solution

| Sample | n | k |
|---|---|---|
| P0L5 in pH 3 | 0.61 ± 0.11 | 0.80 ± 0.10 |
| P0L5 in pH 7 | 0.57 ± 0.07 | 0.79 ± 0.17 |

The data in Table 6 was calculated from the dynamic curve, in FIG. 16 according to the empirical equation shown above. The value of n for P0L5 in both pH 3 and pH 7 were greater than 0.5, indicating that the nature of water diffusion into the hydrogel was non-Fickian. This means that the diffusion and relaxation of hydrogel networks were comparable in relation to their contribution to the swelling ability of hydrogel. Such results are similar to the studies on swelling properties of chitosan (n=0.53 in D.I. water) or dextran derivative (n=0.655) based gels in other literatures (W C Lin et al, 2005; H K Can et al, 2005).

The diffusion nature of water in hydrogel reflects the transportation properties of other small molecules solute in this polymeric matrix. This shows that the hydrogel may be used in drug-delivery.

### (b) Water Content of Hydrogels

The water content of each hydrogel sample was calculated by conducting the DSC analysis of the samples from −15° C. to 15° C. with reference to pure water. Table 7 shows the results of the water content in LCMA hydrogel or LCMA/PEGDA hydrogels. In Table 7, EWC refers to the equilibrium water content, W_{f} refers to free water, W_{fb} refers to freezing bound water, W_{b} refers to non-freezing bound water, Q_{endo} refers to the heats of fusion in the hydrogel and Q_{f} refers to the heats of fusion in pure water.

It was found that the equilibrium water content significantly decreased as the ratio of PEGDA in the hydrogel increased with the concentration of remaining LCMA constant (P0L5 (87.2%), P5L5 (74.7%), P10L5 (46.2%)). Interestingly, although P10L5 has the lowest EWC, probably due to its compact hydrogel structure so that less water could travel through the network, its non-freezing bound water (W_{b}) (22.8%) tremendously boosted to more than twice than that of P0L5 hydrogel (9.5%). This indicated that more water participated in forming hydrogen bonds with the polymeric network, and it also might be attributed to the hydrophilic property of PEGDA. From the values of (W_{f}+W_{fb}), it was concluded that the tendency of the reduction of EWC by enhancing PEGDA content was mainly caused by less free water which was sustained in the denser hydrogel.

TABLE 7
Content of water states in hydrogels

| Hydrogel | EWC | W_{f} + W_{fb} | Wb |
|---|---|---|---|
| Samples | (Wₜ, %) | (Q_{endo}/Q_{f})(%) | (%) |
| P0L5 | 96.9 ± 0.2 | 87.2 ± 1.8 | 9.5 ± 1.8 |
| P5L5 | 82.9 ± 2.0 | 74.7 ± 3.4 | 8.2 ± 1.8 |
| P10L5 | 69.0 ± 0.6 | 46.2 ± 2.4 | 22.8 ± 2.0 |

FIG. 17 shows the DSC melting thermogram of the freezing water in pure water and of swollen hydrogels with different component concentrations. There was only one sharp peak starting from 0° C. in pure water, meaning that only one state of water existed in pure water. In the case of P0L5 hydrogel, there were two transitions, the lower melting temperature attributed to the phase transition of freezing bound water (H B Lee et al, 1975). The transition peak of freezing bound water content (small sharp peak in the left of main peak) became more significant as the amount of LCMA increased in the hydrogels. Similarly, the freezing bound water's percentage increased with more PEGDA content in the copolymeric hydrogel because PEGDA is a hydrophilic polymer that can form hydrogen bonds with water. Notably, the heat flow of freezing bound water was almost equivalent to the value of normal freezing water in P10L5, indicating that the heat flow rate of the two peaks are almost of the same intensity in the thermograms.

### (c) Morphology Observation of Hydrogels

The surface and upper interior structure of the hydrogels was viewed under

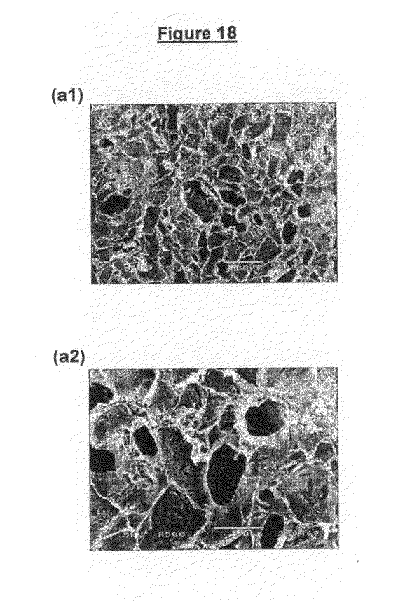

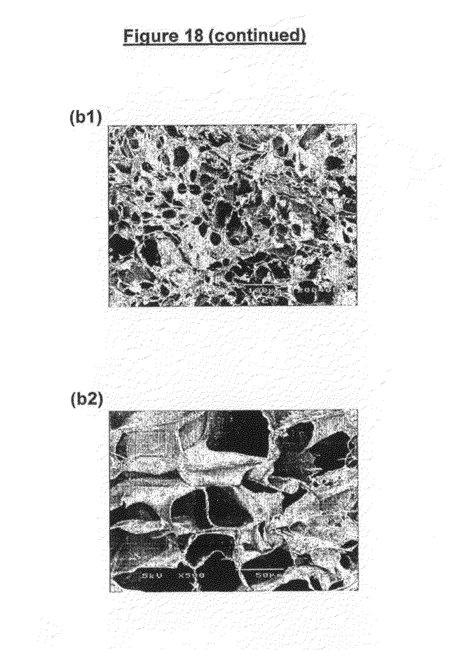

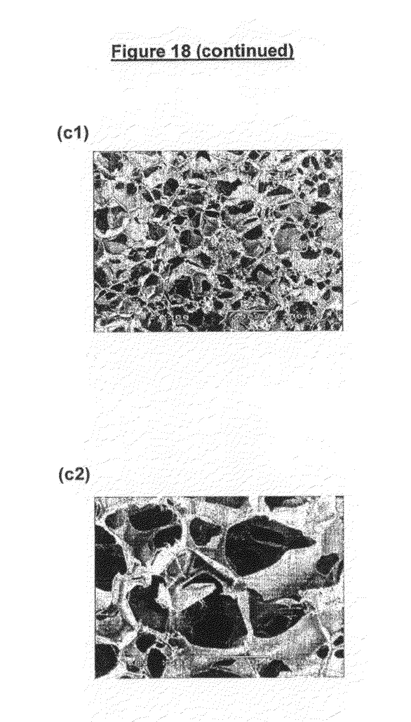

SEM in its dried state. FIG. 18 (a1 to c3) shows six SEM photomicrographs of the morphology of three freeze-dried hydrogels with different concentration of PGDA and LCMA (P0L5, P5L5, and P10L5). All selected photomicrographs presented three-dimensional network. In comparison with the same magnification of the three different hydrogels, the inter-connective channels of the hydrogel without PEGDA were not as small as the copolymerized hydrogel shown in FIG. 18 (b1 to c2). Such differences might be interpreted by the higher concentration of precursor solution resulting in a more compact polymeric structure. Some random cracks were observed on the wall of cells and this was probably due to the poor number of crosslinks leading to poor strength of connective ridges in the pure LCMA hydrogel that was destroyed during vacuum freezing-drying. The size of the micropores was estimated to be from 10 to 70 μm. Such porous scales may accommodate cells and its further growth, or biodegradable vehicles to allow the transport of drugs in various pH media.

### (d) Drug Release Characterization of Hydrogels and Xerogels

Vitamin B₁₂ (VB₁₂) (cyanocobalamin) is a water-soluble vitamin which is dark red coloured with an essential role for the growth and health of humans (S E Lester, 1965). There were several studies involving sustaining and releasing of VB₁₂ in polymer hydrogel. Luo et al., 2001, studied the transportation properties of VB₁₂ from the synthesized hollow fiber membrane based on copolymer of 2-hydroxyethyl methacrylate and methyl methacrylate. The release behavior of VB₁₂ was also investigated in swollen hydrogels made from poly(N-vinyl-2-pyrrolidone) crosslinked with polyacrylamide (R Dengre et al, 2000). The permeability of VB₁₂ through chitosan/PVA blended hydrogel membranes have been explored with the authors finding that the transportation ability increased linearly with the chitosan content in the hydrogel (J M Yang, 2004).

Generally, there are two methods to load drug into hydrogels to render them as a drug delivery system. The first is to combine the drug and monomer prior to polymerization of the hydrogel precursor; the other is post-immersion of the hydrogel in a saturated drug solution for the drug to permeate and be sustained in hydrogels. The latter method is preferred because the drug properties are not subjected to the polymerization process and no subsequent purification of drug-loaded hydrogel is required (S W Kim, 1992). Therefore, in this example, soaking the dehydrated photocrosslinked hydrogel in high concentration of VB₁₂ solution to load the model drug was chosen.

Table 8 shows the loading capacity of VB₁₂ in LCMA xerogel or LCMA/PEGDA xerogel. The photocrosslinked LCMA hydrogel was capable of hosting approximately 30% its dry mass of VB₁₂, significantly more than the copolymer of LCMA and PEGDA. This was attributed to its higher swelling ability and larger interior pore sizes, a function of the lower number of crosslinks in the hydrogel network. In contrast, only about 2.5% VB₁₂ was incorporated into PEGDA to LCMA (P10L5). The loading capacity was increased to 5.2% when the amount of PEGDA was reduced (P5L5). From the results of the loading capacity of hydrogel, it can be concluded that the VB₁₂ loading amount is controllable by regulating the hydrogel composition.

TABLE 8
Loading capacity of Vitamin B₁₂ in xerogel

| | Sample | | |
|---|---|---|---|
| | P0L5 | P5L5 | P10L5 |
| Loading Capacity | 30.73 ± 0.50 | 5.2 ± 0.02 | 2.45 ± 0.07 |
| (%, mg/mg) | | | |

### (i) Dynamic Releasing Profile

The release profile of VB₁₂ of the xerogels and hydrogels are shown in FIGS. 19-20 and 21-22 respectively. The diagrams were plotted as the concentration ratio, of which the concentration of VB₁₂ at designated time (Mₜ) to the final total concentration (M_{inf}) was expressed as Mₜ/M_{inf}, to interval time.

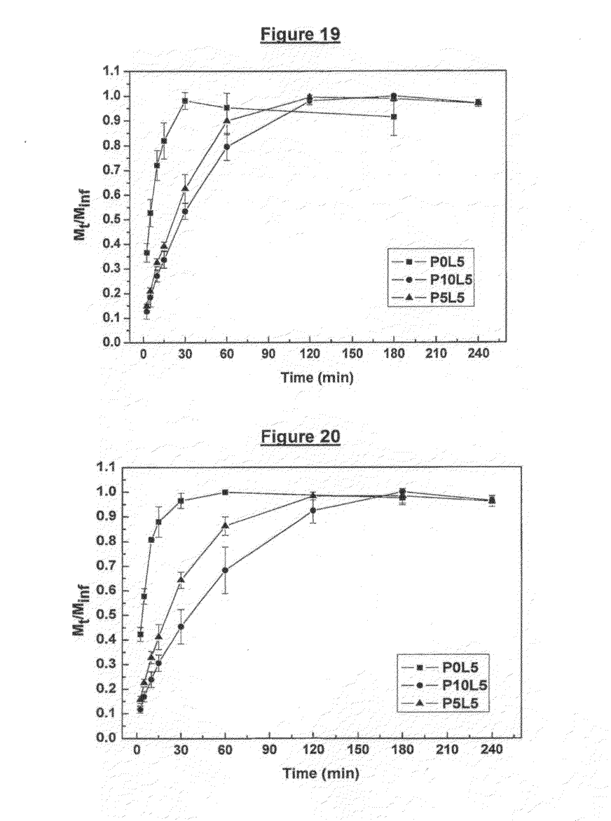

Referring to FIGS. 19 and 20, it was found that the LCMA xerogel (P0L5) released VB₁₂ much faster than the xerogels containing PEGDA in both water and HCl solution which were both about 30 min. In contrast, P5L5 and P10L5 only completely released VB₁₂ over 120 min. Hence, the release profile of the three xerogels have the following order: P0L5>P5L5>P10L5. The releasing rate of P0L5 xerogel compared to P5L5 or P10L5 being about 3.5 times faster shows that swelling ratio decreases with increasing degrees of crosslinking (P J Flory, 1953). Since the degree of crosslinking controls the diffusion and transport of small molecules through the polymeric network, the looser structure of P0L5 results in a fast exchange between water and VB₁₂. Conversely, the complex and compact morphology of P5L5 and P10L5 hydrogels gives rise to slower water permeation rates leading to a slower exchange and release of VB₁₂ in the copolymers.

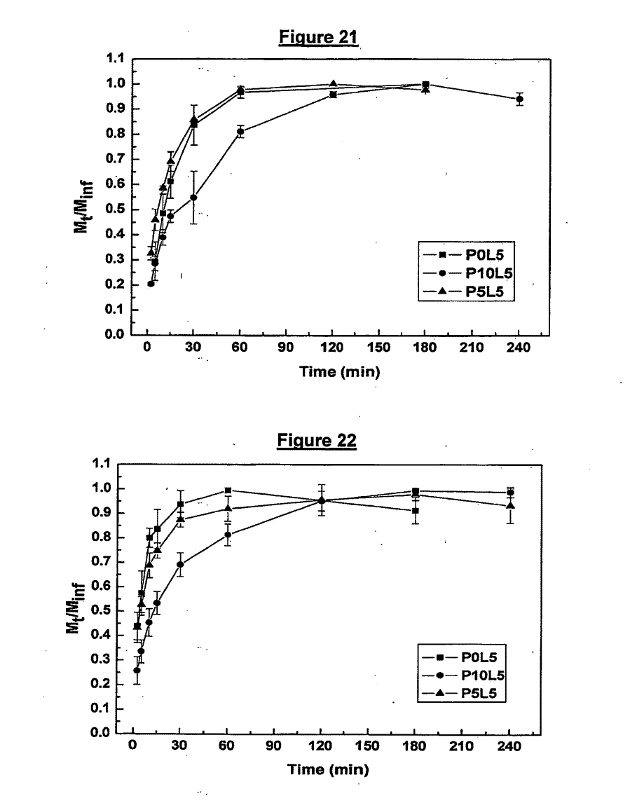

The release feature of VB₁₂ from the hydrogels is shown in FIGS. 21 and 22. The release mechanism of the model drug from the hydrogel involved exclusively the exchange of water molecules and drug molecules, instead of additional factor of swelling behavior in the case of xerogels because the hydrogel had reached its water equilibrium state before drug release evaluation. The three types of hydrogels all released VB₁₂ in less than 3 hours in both water and HCl solution.

It took approximately 60 min for P0L5 to totally release VB₁₂, and 180 min for P5L5 and P10L5. This was almost double the time of the corresponding xerogels. Furthermore, the released drug amounts in the first 10 min were not as rapid as that in the xerogels. This was because the hydrogel was at its equilibrium state and no network expansion was involved upon immersion in the release media. Hence, the release of VB₁₂ would be much steadier from hydrogel in water or acidic media.

All hydrogels were found to be colorless at the endpoint of each examination, i.e. VB₁₂ concentration did not increase any more, indicating that the loaded drug could almost completely be released into media. Such ability to entirely release VB₁₂ would be useful and important in designing drug carriers for delivering hydrophilic drugs with respect to controlling the total drug loading amount as well as the final drug concentration in the body. This is because the effective drug dose released from a drug-releasable system that incompletely releases its drug content could give rise to overdose issues. If the drug delivery system did not totally release the drug at the prescribed therapeutic time, it is reasonable to conclude that when the drug carrier eventually degrades in the body and releases the residual drug, an overdose may arise, that may be detrimental to the patient.

### (ii) Release Mechanism of Model Drug

The diffusion behavior of the model drug released from the polymeric network was analyzed using the empirical equation of:

$$\frac{{M}_{t}}{{M}_{inf}} = k {t}^{n}$$

where Mₜ/M_{inf} is the fractional drug release, Mₜ is the mass, of drug release at time t and M_{inf} is the mass of drug release as time approaches infinity. n is the diffusional exponent characteristic of the release mechanism, k is a constant incorporating the characteristics of the macromolecular network system and drug. The exponential relation is valid for the first 60% of the fractional release. Hence, the data in Tables 9 and 10 was computed based on results of Mₜ/M_{inf}<0.6. When n is equal to 0.5, the case is called Fickian diffusion; it is non-Fickian diffusion if n is between 0.5 and 1.0 (P L Ritger and N A Peppas, 1987, release I; P L Ritger and N A Peppas, 1987, release II).

Tables 9 and 10 show the results of k and n of VB₁₂ released from xerogels in water and HCl. The value of n was almost equal to 0.5 for VB12 released from P10L5 xerogel in water or 0.1M HCl solution. This indicated that the transportation mechanism of solute or drugs from this xerogels followed the Fickian diffusion that can be expressed as Mₜ/M_{inf}=k₁t^{1/2} in water, and Mₜ/M_{inf}=k₂t^{1/2} in 0.1M HCl solution. k₁ is approximately 0.6 in water and k₂ is about 0.7 in HCl. However, for P5L5 where the copolymer contains less PEGDA, the release mechanism deviates from the Fickian diffusion principle. The n value of P5L5 was 0.59 in water and 0.56 in acidic solution.

Furthermore, it was found that n was less than 0.5 when drug was released from the homopolymer of LCMA. This could probably be attributed to the shape of the P0L5 xerogel that was slightly changed to a cylindrical shape. During swelling in water or HCl solution the low crosslinking density resulted in volume expansion of its dimension that was much larger in the release medium. However, the value of n can reduce to the range of 0.45 and 0.89 in the case of cylindrical sample that supports the above prediction of the result of P0L5 (P L Ritger and N A Peppas, 1987, release II).

TABLE 9
Release parameters of VB12 release from xerogels in water (37° C.)

| | Milli-Q Water | | |
|---|---|---|---|
| Sample ID | n | K | R² |
| P0L5 | 0.45 ± 0.06 | 1.59 ± 0.22 | 0.9929 |
| P5L5 | 0.59 ± 0.04 | 0.95 ± 0.07 | 0.9964 |
| P10L5 | 0.52 ± 0.02 | 0.62 ± 0.10 | 0.9927 |

TABLE 10
Release parameters of VB₁₂ release from xerogels in HCl (37° C.)

| | 0.1M HCl | | |
|---|---|---|---|
| Sample ID | n | k | R² |
| P0L5 | 0.39 ± 0.09 | 1.60 ± 0.23 | 0.9829 |
| P5L5 | 0.56 ± 0.03 | 0.92 ± 0.02 | 0.9987 |
| P10L5 | 0.50 ± 0.02 | 0.73 ± 0.06 | 0.997 |

### (e) Conclusion

The new hydrogel synthesised by photocrosslinking of polyethylene glycol) diacrylate and chitin methacrylate having the formula:

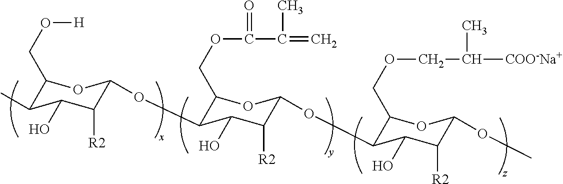

wherein:
x, y, and z are integers and each x, y or z is ≧1; and
each R₂ is the same or different, selected from the group consisting of:

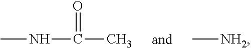

was prepared in an aqueous system. The chitin methacrylate had a degree of N-acetylation equal to or greater than 80%. Example 9 shows that the new hydrogel held a porous structure to be suitable for transporting drug or bioactive agents to a subject, such as a human body. The swelling behavior allowed the hydrogel to be particularly useful in gastro-intestine system. The release profile of the model drug demonstrated the liberating manner of model drug varies with the environmental pH value but results in the total release of the drug.

### REFERENCES

1. B. D. Boyan, C. H. Lohmann, D. D. Dean, V. L. Sylvia, D. L. Cochran, Z. Schwartz. Mechanism involved in osteoblast response to implant surface morphology. Annual Review of Materials Research, 2001, 31: 357-371
2. Crank J. The mathematics of diffusion. Oxford, UK: Clarendon Press; 1978. p 239
3. D Wang, Christopher G Williams, Fan Yang and Jennifer H Elisseeff, Advanced Functional Materials, 2004, 14(2):1152-1159
4. G. A. F. Roberts. Chitin chemistry, The Macmillian Press Ltd, London, 1992. p 249-254
5. G. Cardenas, G. Cabrera, E. Taboada, S. P. Miranda. Chitin characterization by SEM, FTIR, XRD, and 13C cross polarization/mass angle spinning NMR. J. Appl. Polym. Sci., 2004, 93: 1876-1885
6. G. Ciapetti, E. Cenni, L. Pratelli and A. Pizzoferrato. In Vitro evaluation of cell/biomaterial interaction by MTT assay. Biomaterials, 1993, 14 (5): 359-364
7. H. B. Lee, M. S. Jhon, J. D. Andrade. Nature of water in synthetic hydrogels. I. Dilatometry, specific conductivity, and differential scanning calorimetry of polyhydroxyethyl methacrylate. J Colloid Interface Sci, 1975, 51: 255-231
8. H. K. Can, B. K. Denizli, A. Guner, Z. M. O. Rzaev. Effect of functional crosslinking agents on preparation and swelling properties of dextran hydrogels. Carbohydrate Polym, 2005, 25: 51-56
9. ISO/EN 10993-5. Biological evaluation of medical devices—Part 5: tests for in vitro cytotoxicity
10. J. A. Rupley, The hydrolysis of chitin by concentrated hydrochloric acid, and the preparation of low-molecular-weight substrates for lysozyme. Biochimica Et Biophysica Acta, 1964 83: 245
11. J. Brugnerotto, J. Lizardi, F. M. Goycoolea, W. Arguelles-Monal, J. Desbrieres, M. Rinaudo. An infrared investigation in relation with chitin and chitosan characterization. Polymer, 2001, 42: 3569-3580 J. M. Yang, W. Y. Su, T. A. Leu, M. C. Yang. Evaluation of chitosan/PVA blended hydrogel membranes. J Membrane Sci, 2004, 236: 39-51
12. K. S. Chow, E. Khor. New flourinated chitin derivatives: synthesis, characterization and cytotoxicity assessment. Carbohydrate Polymers, 2002, 47: 357-363
13. K. Tomihata and Y. Ikada. In vitro and in vivo degradation of films of chitin and its deactylated derivatives. Biomaterials, 1997, 18: 567-575
14. M. B. Ahmad, M. B. Huglin. DSC studies on states of water in crosslinked Poly(methyl methacrylated-co-N-vinyl-2-pyrrolidine) Hydrogels. Polymer International, 1994, 33: 273-277
15. O. Carmen, V. Blanca, S. R. Julio. Hydrophilic acrylic biomaterials derived from vitamin E with antioxidant properties. Journal of Biomedical Materials Research, 1999, 45: 184-191
16. P. J. Flory. Principles of polymer chemistry, Cornell University Press, Ithaca and London, 1953, p 581-590
17. P. L. Ritger, and N. A. Peppas. A simple equation for description of solute release I. Fickian and non-fickian release from non-swellable devices in the form of slabs spheres cylinders or discs. J Controlled Release, 1987, 5: 23-36
18. P. L. Ritger, and N. A. Peppas. A simple equation for description of solute release II. Fickian and anomalous release from swellable devices. J controlled Release, 1987, 5: 37-42
19. R. A. A. Muzzarelli, M. Guerrieri, G. Goteri, C. Muzzarelli, T. Armeni, R. Ghiselli, M. Cornelissen. The biocompatibility of dibutyryl chitin in the context of wound dressings. Biomaterials, 2005, 26: 5844-5854
20. R. Dengre, M. Bajpai, S. K. Bajpai. Release of vitamin B₁₂ from poly(N-vinyl-2-pyrrolidone)-crosslinked polyacrylamide hydrogels: A kinetic study. J Appl Poly Sci. 2000, 76: 1706-1714
21. S. C. Moldoveanu, Analytical Pyrolysis of Natural Organic Polymers, Techniques and Instrumentation in Analytical Chemistry. Vol. 20, Elsevier, Amsterdam, 1998
22. S. H. Kim, C. C. Chu. Synthesis and characterization of dextran-methacrylate hydrogels and structural study by SEM. Journal of Biomedical Materials Research, 2000, 49: 517-527
23. S. H. Pangburn, P. V. Trescony and J, Heller. Lysozyme degradation of partially deactylated chitin, its film and hydrogels. Biomaterials, 1982, 3: 105-108
24. S. J. Kim, C. K. Lee, S. I. Kim. Characterization of the water state of hyaluronic acid and poly(vinyl alcohol) interpenetrating polymer networks. Journal of Applied Polymer Science, 2004, 92: 1467-1472
25. S. N. Khalid, F. Agnely, N. Yagoubi, J. L. Grossiord, G. Couarraze. Water state characterization, swelling behavior, thermal and mechanical properties of chitosan based network. European Journal of Pharmaceutical Science, 2002, 15: 425-432
26. S. Tanodekaew, M. Prasitsilp, S. Swasdison, B. Thavornyutikarn, T. Pothsree, R. Pateepasen. Preparation of acrylic grafted chitin for wound dressing application. Biomaterials, 2004, 25: 1453-1460 S. E. Lester. Vitamin B₁₂. John Wiley 8, Son Inc. 1965, 3^{rd} Ed. p 144-152
27. S. W. Kim, Y. H. Bae and T. Okano. Hydrogels: swelling, drug loading and release. Pharmaceutical Res. 1992, 9(3): 283-290
28. T. Coviello, M. Grassi, A. Palleschi, G. Bocchinfuso, G. Coluzzi, F. Banishoeib, F. Alhaique. A new scleroglucan/borax hydrogel: swelling and drug release studies. Intern J. Pharm. 2005, 289, 97-107
29. W. C. Lin, D. G. Yu, M. G. Yang. pH-sensitive polyelectrolyte complex gel microspheres composed of chitosan/sodium tripolyphosphate/dextran sulfate: swelling kinetics and drug delivery properties. Colloids and Surfaces B: Biointerfaces, 2005, 44: 143-151
30. Y. Liu, M. B. Huglin. Observations by DSC on bound water structure on some physically crosslinked hydrogels. Polymer International, 1995, 37: 63-67
31. Y. Luo, P. D. Dalton, M. S. Shoichet. Investigating the properties of novel poly(2-hydroxyethyl methacrylate-co-methyl methacrylate) hydrogel hollow fiber membranes. Chem. Mater. 2001, 13: 4087-4093
32. Z. B. Zhang and C. L. Mccormick. Structopendant unsaturated cellulose esters via acylation in homogeneous lithium chloride/N,N-dimethylacetamide solutions. J. Appl. Polym. Sci., 1997, 66: 293-305

1. A composition comprising:
(a) at least one viscosity-enhancing agent; and
(b) at least one macromonomer.

2. The composition according to claim 1, wherein the composition is a bone cement composition and/or a dental cement composition.

3. The composition according to claim 1, wherein the at least one macromonomer is a cross-linking macromonomer.

4-5. (canceled)

6. The composition according to claim 1, wherein the at least one viscosity-enhancing agent has an average molecular weight equal to or greater than 5000 Da.

7. The composition according to claim 1, wherein the at least one viscosity-enhancing agent is selected from the group consisting of: derivatives of chitin, chitosan, cellulose, dextran, collagen, hyaluronic acid and starch.

8. The composition according to claim 1, wherein the at least one viscosity-enhancing agent is selected from the group consisting of: chitin methacrylate, dextran methacrylate, chitosan itaconylate, carboxymethyl chitin, methacrylate hyaluronan, poly(vinyl alcohol), polyethylene oxide, derivatives thereof, and a mixture thereof.

9. The composition according to claim 1, wherein the at least one viscosity-enhancing agent is chitin methacrylate having a structure: 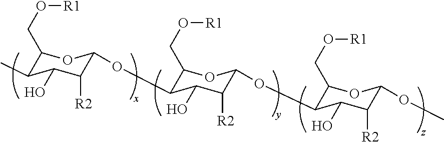
wherein:
x, y, and z are integers and each x, y or z is ≧1;
each R₁ is the same or different, selected from the group consisting of: 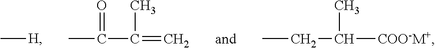
wherein M is a metal; and
each R₂ is the same or different, selected from the group consisting of:

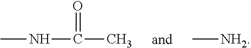

10. The composition according to claim 9, wherein at least one R₁ is

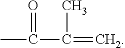

11. The composition according to claim 9, wherein M is a monoelectropositive metal.

12. (canceled)

13. The composition according to claim 9, wherein the viscosity-enhancing agent is: 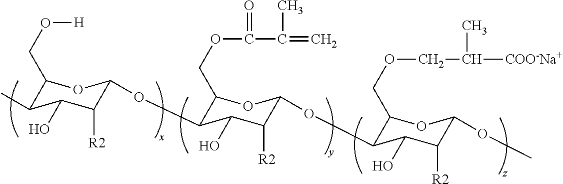
wherein:
x, y, and z are integers and each x, y or z is ≧1; and
each R₂ is the same or different, selected from the group consisting of:

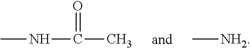

14. The composition according to claim 9, wherein the viscosity-enhancing agent is: 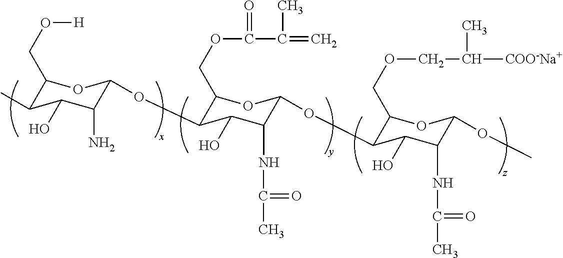
wherein, x, y, and z are integers and each x, y or z is ≧1.

15. The composition according to claim 1, further comprising at least one solvent, osteoconductive material, radiopaque material, polymerization initiator, polymerization accelerator, polymerization inhibitor, bioactive agent, or a mixture thereof.

16-32. (canceled)

33. The composition according to claim 1, for use in medicine or dentistry.

34. (canceled)

35. The composition according to claim 1, for use in periodontal and/or orthopedic applications.

36-40. (canceled)

41. A method of bone filling, bone repairing, bone implanting and/or joining bone implants, comprising applying a composition according to claim 1 to a bone site of a subject.

42. A method of pulp capping, root perforation repair, root-end filling, management of root fracture, root canal filling and/or temporary filling, wherein the method comprises applying a composition according to claim 1 to a tooth and/or gum of a subject.

43. (canceled)

44. The composition according to claim 1 comprised in a bone implant and/or dental implant.

45-118. (canceled)
